(19) United States
(12) Patent Application Publication
(10) Pub. No.: US 2020/0208184 A1
(43) Pub. Date: Jul. 2, 2020

(54) Methods of Producing 5-Ketofructose

(71) Applicants: RWTH Aachen, Aachen (DE); Forschungszentrum Jülich GmbH, Jülich (DE); Rheinische Friedrich-Wilhelms-Universität Bonn, Bonn (DE)

(72) Inventors: Elena Herweg, Köln (DE); Jochen Büchs, Aachen (DE); Michael Bott, Jülich (DE); Ines Kiefler, Mönchengladbach (DE); Uwe Deppenmeier, Lohmar (DE); Konrad Kosciow, Bonn (DE); Anna Siemen, Köln (DE)

(73) Assignees: RWTH Aachen, Aachen (DE); Forschungszentrum Jülich GmbH, Jülich (DE); Rheinische Friedrich-Wilhelms-Universität Bonn, Bonn (DE)

(21) Appl. No.: 16/622,439

(22) PCT Filed: Jun. 13, 2018
(86) PCT No.: PCT/EP2018/065724

§ 371 Date: Dec. 13, 2019

(30) Foreign Application Priority Data
Jun. 13, 2017 (EP) 17175730.5

(51) Int. Cl. C12P 19/02

(52) U.S. Cl. CPC C12P 19/02; C12Y 101/99011

(57) ABSTRACT

A method of producing 5-ketofructose using an acetic acid bacterium which is genetically modified to express a fructose dehydrogenase as well as to microorganisms which can be used in said method.

### FIELD OF THE INVENTION

The present invention relates to a method of producing 5-ketofructose using an acetic acid bacterium which is genetically modified to express a fructose dehydrogenase as well as to microorganisms which can be used in said method.

### BACKGROUND OF THE INVENTION

Low-calorie sweeteners provide consumers with both psychological and physiological benefits. For example, low-calorie sweeteners are believed to be effective for weight maintenance, weight reduction, management of diabetes, reduction of dental caries, and reduction in the risks associated with obesity. However, they are also used as part of an overall healthy lifestyle. Low-calorie sweeteners which are present in currently available food products include aspartame, acesulfame K, sugar alcohols, D-tagatose, steviol glycosides and saccharine.

However, at present there is no natural, low-calorie sweetener which has good sensoric properties and which cannot be metabolized by intestinal bacteria. For example, the sweetener composition Stevia obtained from the plant Stevia rebaudiana has a licorice-like taste and is therefore not suitable for all applications.

In contrast, 5-ketofructose cannot be metabolized by intestinal bacteria, has a natural sweetness and a high sweetening intensity in the same range as fructose.

DE 10 2007 026 713 discloses that 5-ketofructose produced in vitro using a purified 5-D-fructose dehydrogenase can be used as a sweetener. The 5-ketofructose may also be used as a starting material for the synthesis of pyrrolidine aza sugars which can be used pharmaceutically as glycohydrolase inhibitors (Baxter and Reitz (1994) J. Org. Chem. 59: 3175-3185).

Methods for producing 5-ketofructose have been described in the art. For example, L-sorbose can be converted to 5-ketofructose in vitro using the purified enzyme pyranose-2-oxidase, yielding up to 180 g/l 5-ketofructose (Schneider et al. (2012) Biotechnol. Bioeng. 109: 2941-2945). For regenerating the co-substrate oxygen catalases have to be used. Since the enzymes required in this process need to be produced and purified, this method is expensive and time-consuming.

Ameyama et al. (1981) J. Bacteriol. 145(2): 814-823 describes the purification and characterization of a fructose dehydrogenase from Gluconobacter industrius and JP 2010 148 368 describes a fructose dehydrogenase from Gluconobacter frateurii and its recombinant production.

Kawai et al. (2012) Appl. Environm. Microbiol. 79(5): 1654-1660 discloses the recombinant expression of a fructose dehydrogenase in Gluconobacter oxydans. According to this document the fructose dehydrogenase can be used to determine the fructose content.

U.S. Pat. No. 3,206,375 A and GB 981 273 describe the conversion of fructose to 5-ketofructose in different wild-type strains of the genus Acetobacter, yielding 10 to 16 g purified 5-ketofructose from 100 g/l fructose. However, this method is inefficient, since only a small part of the fructose is converted slowly to 5-ketofructose. Further, the wild-type strains produce overflow metabolites such as acetate, further reducing the product yield.

Hence, there is still a need for an efficient method of producing 5-ketofructose.

### SUMMARY OF THE INVENTION

The present inventors have surprisingly found that 5-ketofructose can be produced efficiently in acetic acid bacteria which are genetically modified to express a recombinant fructose dehydrogenase, using fructose, glucose, saccharose or starch as the starting material for the production of 5-ketofructose.

Accordingly, the present invention relates to a method for producing 5-ketofructose in an acetic acid bacterium, comprising the steps of:

a) contacting a first acetic acid bacterium which is genetically modified to express a fructose dehydrogenase with a solution comprising a carbohydrate in fed-batch mode; and

b) isolating the 5-ketofructose.

The first acetic acid bacterium may contain a first expression vector comprising a nucleic acid sequence which encodes a fructose dehydrogenase.

The acetic acid bacterium may be from the genus Gluconobacter, preferably the acetic acid bacterium is Gluconobacter oxydans.

In one embodiment, the expression vector further comprises a promoter region from Gluconobacter which may be from a gene encoding a ribosomal protein.

In one embodiment step (a) comprises incubating a purified acetic acid bacterium with a solution comprising said carbohydrate in fed-batch mode.

In another embodiment step (a) comprises culturing the acetic acid bacterium in a culture medium comprising said carbohydrate in fed-batch mode.

The fed-batch mode may not result in limiting conditions with respect to said carbohydrate.

In one embodiment the carbohydrate is fructose.

The carbohydrate may be fructose and the concentration of fructose in the culture medium is 30 to 300 g/l.

The carbohydrate may be fructose and the cells are fed with a solution comprising 300 to 1,500 g/l fructose.

In another embodiment the carbohydrate is glucose and the glucose is converted to fructose by the action of a glucose isomerase and wherein the acetic acid bacterium lacks expression of a membrane-bound glucose dehydrogenase.

An isolated glucose isomerase may be added to the solution comprising glucose.

The first acetic acid bacterium may be genetically modified to express a glucose isomerase.

The first acetic acid bacterium may comprise a second expression vector comprising a nucleic acid sequence which encodes a glucose isomerase.

In another embodiment a second acetic acid bacterium genetically modified to express a glucose isomerase is contacted with said solution comprising glucose.

The second acetic acid bacterium may contain an expression vector comprising a nucleic acid sequence which encodes a glucose isomerase In another embodiment the carbohydrate is sucrose and the sucrose is converted to fructose by the action of a glucose isomerase and an invertase and wherein the acetic acid bacterium lacks expression of a membrane-bound glucose dehydrogenase.

An isolated glucose isomerase and/or invertase may be added to the solution comprising sucrose.

The first acetic acid bacterium may be genetically modified to express a glucose isomerase and/or an invertase.

In one embodiment the first acetic acid bacterium comprises a second expression vector comprising a nucleic acid sequence which encodes a glucose isomerase and/or a third expression vector comprising a nucleic acid sequence which encodes an invertase.

In another embodiment a second acetic acid bacterium genetically modified to express a glucose isomerase and/or an invertase is contacted with said solution comprising sucrose.

The second acetic acid bacterium may contain an expression vector comprising a nucleic acid sequence which encodes a glucose isomerase and/or a nucleic acid sequence which encodes an invertase.

In one embodiment a second acetic acid bacterium genetically modified to express a glucose isomerase and a third acetic acid bacterium genetically modified to express an invertase are contacted with said solution comprising sucrose.

The second acetic acid bacterium may contain an expression vector comprising a nucleic acid sequence which encodes a glucose isomerase and the third acetic acid bacterium contains an expression vector comprising a nucleic acid sequence which encodes an invertase.

In another embodiment the carbohydrate is starch and the starch is converted to fructose by the action of an amylase, a pullulanase and a glucose isomerase and wherein the acetic acid bacterium lacks expression of a membrane-bound glucose dehydrogenase.

An isolated glucose isomerase and/or amylase and/or pullulanase may be added to the solution comprising starch.

The first acetic acid bacterium may be genetically modified to express a glucose isomerase, an amylase and/or a pullulanase.

The first acetic acid bacterium may comprise a second expression vector comprising a nucleic acid sequence which encodes a glucose isomerase and/or a third expression vector comprising a nucleic acid sequence which encodes an amylase and/or a fourth expression vector comprising a nucleic acid sequence which encodes a pullulanase.

In an alternative embodiment a second acetic acid bacterium genetically modified to express a glucose isomerase and/or a third acetic acid bacterium genetically modified to express an amylase and/or a fourth acetic acid bacterium genetically modified to express a pullulanase is contacted with said solution comprising starch.

The second acetic acid bacterium may contain an expression vector comprising a nucleic acid sequence which encodes a glucose isomerase, the third acetic acid bacterium contains an expression vector comprising a nucleic acid sequence which encodes an amylase and the fourth acetic acid bacterium contains an expression vector comprising a nucleic acid sequence which encodes a pullulanase.

In one embodiment the acetic acid bacterium lacks expression of both a membrane-bound and a soluble glucose dehydrogenase.

The acetic acid bacterium may comprise a genetic modification causing a leaky outer membrane which may be a deletion of the tolB gene.

The present invention also relates to a method for producing a sweetener composition, comprising producing 5-ketofructose by the method disclosed herein and preparing a sweetener composition comprising 5-ketofructose.

The sweetener composition may be a powder, solution, granule or pill.

The present invention also relates to a method for producing a non-alcoholic beverage containing 5-ketofructose, comprising producing 5-ketofructose by the method disclosed herein and adding the 5-ketofructose to a non-alcoholic beverage.

The present invention also relates to a non-alcoholic beverage containing 5-ketofructose.

The present invention also relates to the use of an acetic acid bacterium which is genetically modified to express a fructose dehydrogenase for producing 5-ketofructose.

The acetic acid bacterium may contain a first expression vector comprising a nucleic acid sequence which encodes a fructose dehydrogenase.

The acetic acid bacterium may be Gluconobacter oxydans.

The acetic acid bacterium may be further genetically modified to express a glucose isomerase.

The acetic acid bacterium may comprise a second expression vector comprising a nucleic acid sequence which encodes a glucose isomerase.

The acetic acid bacterium may be further genetically modified to express an invertase.

The acetic acid bacterium may further comprise a third expression vector comprising a nucleic acid sequence which encodes an invertase.

The acetic acid bacterium may be further genetically modified to express an amylase and a pullulanase.

The acetic acid bacterium may further comprise a third expression vector comprising a nucleic acid sequence which encodes an amylase and a fourth expression vector comprising a nucleic acid sequence which encodes a pullulanase.

The present invention also relates to a Gluconobacter oxydans cell containing an expression vector comprising a first nucleic acid sequence which encodes a fructose dehydrogenase and a second nucleic acid sequence which encodes a glucose isomerase.

The expression vector may further comprise a third nucleic acid sequence which encodes an invertase.

The expression vector may further comprise a third nucleic acid sequence which encodes an amylase and/or a fourth nucleic acid sequence which encodes a pullulanase.

The present invention also relates to a Gluconobacter oxydans cell containing a first expression vector comprising a nucleic acid sequence which encodes a fructose dehydrogenase and a second expression vector comprising a nucleic acid sequence which encodes a glucose isomerase.

The cell may further contain a third expression vector comprising a nucleic acid sequence which encodes an invertase.

The cell may further contain a third expression vector comprising a nucleic acid sequence which encodes an amylase and/or a fourth expression vector comprising a nucleic acid sequence which encodes a pullulanase.

The cell may lack expression of a membrane-bound glucose dehydrogenase.

The cell may lack expression of both a membrane-bound or a soluble glucose dehydrogenase.

The cell may further comprise a mutation causing a leaky outer membrane which may be a deletion of the tolB gene.

### BRIEF DESCRIPTION OF THE DRAWINGS

FIG. 1: Conversion of fructose to 5-ketofructose in the G. oxydans wild-type strain and in a recombinant G. oxydans strain expressing fructose dehydrogenase

FIG. 2: Production of 5-ketofructose from fructose in a fed-batch process

FIG. 3: Production of 5-ketofructose from sucrose in a mixed culture of two genetically modified Gluconobacter oxydans strains, wherein the first strain expresses an invertase and the second strain expresses a fructose dehydrogenase

FIG. 4: Production of 5-ketofructose from sucrose in a Gluconobacter strain containing a fructose dehydrogenase complex expressed from a nucleic acid sequence integrated into the genome and a plasmid encoding the extracellular invertase from Zymomonas mobilis

### DETAILED DESCRIPTION OF THE INVENTION

Although the present invention will be described with respect to particular embodiments, this description is not to be construed in a limiting sense.

Before describing in detail exemplary embodiments of the present invention, definitions important for understanding the present invention are given. As used in this specification and in the appended claims, the singular forms of “a” and “an” also include the respective plurals unless the context clearly dictates otherwise. In particular, the term “acetic acid bacterium” typically includes a composition comprising several single acetic acid bacterial organisms. In the context of the present invention, the terms “about” and “approximately” denote an interval of accuracy that a person skilled in the art will understand to still ensure the technical effect of the feature in question. The term typically indicates a deviation from the indicated numerical value of ±20%, preferably ±15%, more preferably ±10%, and even more preferably ±5%. It is to be understood that the term “comprising” is not limiting. For the purposes of the present invention the term “consisting of” is considered to be a preferred embodiment of the term “comprising”. If hereinafter a group is defined to comprise at least a certain number of embodiments, this is meant to also encompass a group which preferably consists of these embodiments only. Furthermore, the terms “first”, “second”, “third” or “(a)”, “(b)”, “(c)”, “(d)” etc. and the like in the description and in the claims, are used for distinguishing between similar elements and not necessarily for describing a sequential or chronological order. It is to be understood that the terms so used are interchangeable under appropriate circumstances and that the embodiments of the invention described herein are capable of operation in other sequences than described or illustrated herein. In case the terms “first”, “second”, “third” or “(a)”, “(b)”, “(c)”, “(d)”, “i”, “ii” etc. relate to steps of a method or use or assay there is no time or time interval coherence between the steps, i.e. the steps may be carried out simultaneously or there may be time intervals of seconds, minutes, hours, days, weeks, months or even years between such steps, unless otherwise indicated in the application as set forth herein above or below.

It is to be understood that this invention is not limited to the particular methodology, protocols, reagents etc. described herein as these may vary. It is also to be understood that the terminology used herein is for the purpose of describing particular embodiments only, and is not intended to limit the scope of the present invention that will be limited only by the appended claims. Unless defined otherwise, all technical and scientific terms used herein have the same meanings as commonly understood by one of ordinary skill in the art.

As discussed above, the present invention is based on the finding that 5-ketofructose can be efficiently produced in an acetic acid bacterium genetically modified to express a fructose dehydrogenase. 5-ketofructose which can be produced from fructose by a dehydrogenation reaction has the following structure:

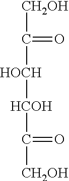

The 5-ketofructose produced by the method of the present invention can be used as a sweetener or for the synthesis of pyrrolidine aza sugars.

In the methods of the present invention the 5-ketofructose is produced in an acetic acid bacterium. Acetic acid bacteria are strictly aerobic gram-negative bacteria which oxidize sugars or ethanol and may produce acetic acid during fermentation. Within the present invention, the term “acetic acid bacterium” is intended to comprise a bacterium from a genus selected from the group consisting of Acetobacter, Acidomonas, Ameyamaea, Asaia, Gluconacetobacter, Gluconobacter, Granulibacter, Kozakia, Neoasaia, Neokomagataea, Saccharibacter, Swaminathania and Tanticharoenia.

Preferably, the acetic acid bacterium is from the genus Gluconobacter. The genus Gluconobacter includes, but is not limited to, the species Gluconobacter albidus, Gluconobacter asaii, Gluconobacter cerevisiae, Gluconobacter cerinus, Gluconobacter frateurii, Gluconobacter japonicus, Gluconobacter kanchanaburiensis, Gluconobacter kondonii, Gluconobacter nephelii, Gluconobacter oxydans, Gluconobacter sphaericus, Gluconobacter thailandicus, Gluconobacter uchimurae and Gluconobacter wancherniae.

More preferably, the acetic acid bacterium is Gluconobacter oxydans. Different strains of Gluconobacter oxydans are available to the skilled person, including Gluconobacter oxydans DSM 4025, Gluconobacter oxydans DSM 3503, Gluconobacter oxydans NBIMCC 1043, Gluconobacter oxydans ATCC 621, Gluconobacter oxydans NBIMCC 902, Gluconobacter oxydans CCM 3607 and Gluconobacter oxydans CCM 1783. Most preferably, Gluconobacter oxydans ATCC 621 is used in the methods of the present invention.

The acetic acid bacterium used in the methods of the present invention is genetically modified to express a fructose dehydrogenase. The term “genetically modified acetic acid bacterium” or “acetic acid bacterium which is genetically modified” as used herein means that an acetic acid bacterium is altered by any suitable genetic means and methods known to the skilled person in order to express a fructose dehydrogenase or to increase the expression of fructose dehydrogenase in the acetic acid bacterium. Methods for genetically modifying acetic acid bacteria are known to the person skilled in the art and are described in the literature (see, e.g., Kallnik et al. (2010) J. Biotechnol. 150: 460-465; Kostner et al. (2013) Appl. Microbiol. Biotechnol. 97: 8341-8349; Kosciow et al. (2014) J. Biotechnol. 189: 27-35; Kiefler et al. (2017) Appl. Microbiol. Biotechnol. in press). They comprise commonly used methods for introducing genetic elements or material into the acetic acid bacterium so as to be contained in the acetic acid bacterium, integrated into the chromosome or extrachromosomally, or the removal or destruction, or modification, of genetic elements or sequences present in the genome of a wild-type acetic acid bacterium. In one embodiment the nucleic acid sequence encoding the fructose dehydrogenase is integrated into the genome of the acetic acid bacterium. In one embodiment the nucleic acid sequence encoding the fructose dehydrogenase is integrated into the genome of the acetic acid bacterium and said acetic acid bacterium further comprises one or more plasmids comprising a nucleic acid sequence which encodes one or more proteins selected from the group consisting of glucose isomerase, invertase, amylase and pullulanase.

The genetically modified acetic acid bacterium can be distinguished from an acetic acid bacterium which is not genetically modified in that it expresses a fructose dehydrogenase which is not naturally expressed in the acetic acid bacterium which is genetically modified. Alternatively, the expression of a fructose dehydrogenase which is naturally expressed in the acetic acid bacterium which is genetically modified is increased in the genetically modified acetic acid bacterium compared to the acetic acid bacterium which is not genetically modified, e.g. by using a promoter which is stronger than the one naturally linked to the fructose dehydrogenase gene. Further, the genetically modified acetic acid bacterium can be distinguished from an acetic acid bacterium which is not genetically modified by the presence of the genetic element which leads to an increased expression of fructose dehydrogenase. If the genetic element leading to an increased expression of fructose dehydrogenase is an expression vector comprising a nucleic acid sequence encoding the fructose dehydrogenase, the genetically modified acetic acid bacterium can be distinguished from an acetic acid bacterium which is not genetically modified by the presence of this expression vector within the cell. For example, it can be determined that the nucleic acid sequence encoding the fructose dehydrogenase is operably linked to regulatory sequences such as a promoter to which it is not naturally linked in the organism from which the nucleic acid sequence encoding the fructose dehydrogenase is derived.

Preferably, the acetic acid bacterium is genetically modified with an expression vector encoding the fructose dehydrogenase.

As used herein, the term “vector” refers to a nucleic acid molecule capable of transporting another nucleic acid to which it has been linked and may be used herein interchangeably with the term “recombinant nucleic acid molecule”. One type of vector is a “plasmid”, which refers to a circular double stranded DNA loop into which additional DNA segments can be ligated. In the present specification, “plasmid” and “vector” can be used interchangeably as the plasmid is the most commonly used form of vector. The term “expression vector” means a vector capable of directing expression of a particular nucleotide sequence in an appropriate host cell. An expression vector comprises a regulatory nucleic acid element operably linked to a nucleic acid of interest, which is—optionally—operably linked to a termination signal and/or other regulatory element.

The term “genetic element” as used herein means any molecular unit which is able to transport genetic information. It accordingly relates to a gene, preferably to a native gene, a chimeric gene, a foreign gene, a transgene or a codon-optimized gene. The term “gene” refers to a nucleic acid molecule or fragment that expresses a specific protein, preferably it refers to nucleic acid molecules including regulatory sequences preceding (5′ non-coding sequences) and following (3′ non-coding sequences) the coding sequence. The term “native gene” refers to a gene as found in nature, e.g. in a wild-type strain of an acetic acid bacterium, with its own regulatory sequences. The term “chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. According to the present invention a “foreign gene” refers to a gene not normally found in the acetic acid bacterium, but that is introduced into the acetic acid bacterium by gene transfer. Foreign genes can comprise native genes inserted into a non-native organism, or chimeric genes. The term “transgene” refers to a gene that has been introduced into the genome by a transformation procedure.

The term “coding sequence” refers to a DNA sequence which codes for a specific amino acid sequence. The term “regulatory sequence” refers to a nucleotide sequence located upstream (5′ non-coding sequences), within, or downstream (3′ non-coding sequences) of a coding sequence, and which influences the transcription, RNA processing or stability, or translation of the associated coding sequence. Regulatory sequences may include promoters, enhancers, translation leader sequences, introns, polyadenylation recognition sequences, RNA processing sites, effector binding sites and stem-loop structures.

The term “promoter” refers to a DNA sequence capable of starting and controlling the expression of a coding sequence or functional RNA. Typically, a coding sequence is located 3′ to a promoter sequence. Promoters may be derived in their entirety from a native gene, or be composed of different elements derived from different promoters found in nature, or even comprise synthetic DNA segments. Typically, since the exact boundaries of regulatory sequences have not been completely defined, DNA fragments of different lengths may have identical promoter activity. It is understood by a person skilled in the art that different promoters may direct the expression of a gene at different stages of development, or in response to different environmental or physiological conditions. Promoters that cause a gene to be expressed in most cell types at most times are commonly referred to as constitutive promoters. On the other hand, promoters that cause a gene to be expressed in specific contexts only, e.g. based on the presence of specific factors, growth stages, temperatures, pH or the presence of specific metabolites etc. are understood as regulable promoters.

The term “3′ non-coding sequences” refers to DNA sequences located downstream of a coding sequence. The term “RNA transcript” refers to the product resulting from RNA polymerase catalyzed transcription of a DNA sequence. The term “mRNA” refers to messenger RNA, i.e. RNA that is without introns and that can be translated into protein by the cell.

The term “operably linked” refers to the association of nucleic acid sequences on a single nucleic acid fragment so that the function of one is affected by the other. In the context of a promoter the term means that the coding sequence is under the transcriptional control of the promoter which regulates the expression of the coding sequence.

Regulatory elements for driving expression of genes in acetic acid bacteria are known to the person skilled in the art and include constitutive promoters of genes encoding ribosomal proteins, preferably the promoter is the p264 or the p452 promoter of Gluconobacter (see Kallnik et al. (2010) J. Biotechnol. 150: 460-465). More preferably, the promoter is the p264 promoter.

Even more preferably, the promoter has a nucleic acid sequence selected from the group consisting of:

a) the nucleic acid sequence according to SEQ ID No. 7;

b) a functional part of the nucleic acid sequence according to SEQ ID No. 7; and

c) a nucleic acid sequence hybridizing under stringent conditions with a complementary sequence of the nucleic acid sequence according to SEQ ID No. 7.

The functional part of the nucleic acid sequence according to SEQ ID No. 7 is able to drive expression of a nucleic acid sequence which is operably linked to it substantially to the same extent as the full-length sequence according to SEQ ID No. 7, i.e. the expression level of the nucleic acid sequence which is linked to the functional part is at least 75%, preferably at least 80% or 85%, more preferably at least 90% or 92% and most preferably at least 95% of the expression level of said nucleic acid sequence when linked to the full-length promoter sequence according to SEQ ID No. 7.

The term “hybridizing under stringent conditions” denotes in the context of the present invention that the hybridization is implemented in vitro under conditions which are stringent enough to ensure a specific hybridization. Stringent in vitro hybridization conditions are known to those skilled in the art and may be taken from the literature (e.g. Sambrook and Russell (2001) Molecular Cloning: A Laboratory Manual, 3rd edition, Cold Spring Harbour Laboratory Press, Cold Spring Harbour, N.Y.). The term “specific hybridization” refers to the circumstance that a molecule, under stringent conditions, preferably binds to a certain nucleic acid sequence, i.e. the target sequence, if the same is part of a complex mixture of, e.g. DNA or RNA molecules, but does not, or at least very rarely, bind to other sequences.

Stringent conditions depend on the circumstances. Longer sequences hybridize specifically at higher temperatures. In general, stringent conditions are chosen such that the hybridization temperature is about 5° C. below the melting point (Tₘ) of the specific sequence at a defined ionic strength and at a defined pH value. Tₘ is the temperature (at a defined pH value, a defined ionic strength and a defined nucleic acid concentration), at which 50% of the molecules complementary to the target sequence hybridize to the target sequence in the state of equilibrium. Typically, stringent conditions are conditions, where the salt concentration has a sodium ion concentration (or concentration of a different salt) of at least about 0.01 to 1.0 M at a pH value between 7.0 and 8.3, and the temperature is at least 30° C. for small molecules (i.e. 10 to 50 nucleotides, for example). In addition, stringent conditions may include the addition of substances, such as, e. g., formamide, which destabilise the hybrids. At hybridization under stringent conditions, as used herein, normally nucleotide sequences which are at least 60% homologous to each other hybridize to each other. Preferably, said stringent conditions are chosen such that sequences which are about 65%, preferably at least about 70%, and especially preferably at least about 75% or higher homologous to each other, normally remain hybridized to each other. A preferred but non-limiting example of stringent hybridization conditions is hybridizations in 6×sodium chloride/sodium citrate (SSC) at about 45° C., followed by one or more washing steps in 0.2×SSC, 0.1% SDS at 50 to 65° C. The temperature depends on the type of the nucleic acid and is between 42° C. and 58° C. in an aqueous buffer having a concentration of 0.1 to 5×SSC (pH value 7.2).

If an organic solvent, e.g. 50% formamide, is present in the above-mentioned buffer, the temperature is about 42° C. under standard conditions. Preferably, the hybridisation conditions for DNA:DNA hybrids are, for example, 0.1×SSC and 20° C. to 45° C., preferably 30° C. to 45° C. Preferably, the hybridisation conditions for DNA:RNA hybrids are, for example, 0.1×SSC and 30° C. to 55° C., preferably between 45° C. and 55° C. The above-mentioned hybridization temperatures are determined, for example, for a nucleic acid which is 100 base pairs long and has a G/C content of 50% in the absence of formamide. Those skilled in the art know how to determine the required hybridization conditions using text books such as those mentioned above or the following textbooks: Current Protocols in Molecular Biology, John Wiley & Sons, N.Y. (1989), Hames and Higgins (publ.) 1985, Nucleic Acids Hybridization: A Practical Approach, IRL Press at Oxford University Press, Oxford; Brown (publ.) 1991, Essential Molecular Biology: A Practical Approach, IRL Press at Oxford University Press, Oxford.

Typical hybridization and washing buffers for example have the following composition:

Pre-hybridization solution: 0.5% SDS
5×SSC
50 mM NaPO₄, pH 6.8
0.1% sodium pyrophosphate
5×Denhardt's solution
100 μg/mL salmon sperm DNA

Hybridization solution: pre-hybridization solution
1×10⁶ cpm/mL probe (5-10 min 95° C.)

20×SSC: 3 M NaCl
0.3 M sodium citrate
ad pH 7 with HCl

50×Denhardt's reagent: 5 g Ficoll
5 g polyvinylpyrrolidone
5 g bovine serum albumin
ad 500 mL aqua destillata

A typical procedure for hybridization is as follows:

Optional: wash blot 30 min in 1×SSC/0.1% SDS at 65° C.

Pre-hybridization: at least 2 h at 50-55° C.

Hybridization: over night at 55-60° C.

| | | | |
|---|---|---|---|
| Washing: | 05 min | 2x SSC/0.1% SDS | hybridization temp. |
| | 30 min | 2x SSC/0.1% SDS | hybridization temp. |
| | 30 min | 1x SSC/0.1% SDS | hybridization temp. |
| | 45 min | 0.2x SSC/0.1% SDS | 65° C. |
| | 5 min | 0.1x SSC | room temperature |

Those skilled in the art know that the given solutions and the presented protocol may be modified or have to be modified, depending on the application.

In the present invention, an acetic acid bacterium is genetically modified to express a fructose dehydrogenase. The fructose dehydrogenase catalyzes the oxidation of fructose to 5-ketofructose. The fructose dehydrogenase may be from Gluconobacter japonicus, Gluconobacter frateurii (see JP 2010 148368), Gluconobacter industrius (see Ameyama et al. (1981) J. Bacteriol. 145(2): 814-823), Gluconobacter albidus, Gluconobacter cerinus, Neokomagataea thailandica, Frateuria aurantia or Tatumella sp. It is a heterotrimeric membrane-bound enzyme which is composed of three subunits which are encoded by separate nucleic acid sequences.

Preferably, the fructose dehydrogenase used in the methods of the present invention is from Gluconobacter japonicus. It is composed of the three subunits FdhS, FdhC and FdhL which are encoded by separate nucleic acid sequences.

The subunit FdhS is preferably encoded by a nucleic acid sequence selected from the group consisting of:
a) a nucleic acid sequence comprising the sequence according to SEQ ID No. 1 or a fragment thereof;
b) a nucleic acid sequence encoding a protein comprising the amino acid sequence according to SEQ ID No. 2;
c) a nucleic acid sequence comprising a sequence which is at least 70% identical to the sequence according to SEQ ID No. 1 or a fragment thereof;
d) a nucleic acid sequence hybridizing under stringent conditions with a complementary sequence of a nucleic acid sequence according to SEQ ID No. 1 or a fragment thereof; and
e) a nucleic acid sequence encoding the same fructose dehydrogenase as any of the nucleic acid sequences of (a) to (d) above, but differing from the nucleic acid sequences of (a) to (d) above due to the degeneracy of the genetic code.

The subunit FdhC is preferably encoded by a nucleic acid sequence selected from the group consisting of:
a) a nucleic acid sequence comprising the sequence according to SEQ ID No. 3 or a fragment thereof;
b) a nucleic acid sequence encoding a protein comprising the amino acid sequence according to SEQ ID No. 4;
c) a nucleic acid sequence comprising a sequence which is at least 70% identical to the sequence according to SEQ ID No. 3 or a fragment thereof;
d) a nucleic acid sequence hybridizing under stringent conditions with a complementary sequence of a nucleic acid sequence according to SEQ ID No. 3 or a fragment thereof; and
e) a nucleic acid sequence encoding the same fructose dehydrogenase as any of the nucleic acid sequences of (a) to (d) above, but differing from the nucleic acid sequences of (a) to (d) above due to the degeneracy of the genetic code.

The subunit FdhL is preferably encoded by a nucleic acid sequence selected from the group consisting of:
a) a nucleic acid sequence comprising the sequence according to SEQ ID No. 5 or a fragment thereof;
b) a nucleic acid sequence encoding a protein comprising the amino acid sequence according to SEQ ID No. 6;
c) a nucleic acid sequence comprising a sequence which is at least 70% identical to the sequence according to SEQ ID No. 5 or a fragment thereof;
d) a nucleic acid sequence hybridizing under stringent conditions with a complementary sequence of a nucleic acid sequence according to SEQ ID No. 5 or a fragment thereof; and
e) a nucleic acid sequence encoding the same fructose dehydrogenase as any of the nucleic acid sequences of (a) to (d) above, but differing from the nucleic acid sequences of (a) to (d) above due to the degeneracy of the genetic code.

A “fragment” of the nucleic acid sequence according to any one of SEQ ID Nos. 1, 3 or 5 is understood to refer to a smaller part of this nucleic acid sequence which consists of a contiguous nucleotide sequence found in SEQ ID No. 1, 3 or 5 and which encodes a protein having the activity of a fructose dehydrogenase when expressed with the other two subunits of fructose dehydrogenase as specified above.

In case the fragment is described to be a fragment of a sequence with a certain degree of sequence identity to a particular sequence, the fragment shall be a fragment of the sequence which has a certain degree of sequence identity to the particular sequence.

Thus, for instance, in expressions like “a nucleic acid sequence comprising a sequence which is at least 70% identical to the sequence according to SEQ ID No. 1, 3 or 5 or a fragment thereof” the “fragment” in the second alternative refers to a fragment of the sequence which sequence is at least 70% identical to the sequence according to SEQ ID No. 1, 3 or 5.

The fragment of SEQ ID No. 1 has a length of at least 300 nucleotides, preferably of at least 320, 340 or 360 nucleotides, more preferably of at least 380, 400 and 420 nucleotides and most preferably of at least 440, 460, 480, 500, 520 or 540 nucleotides. The fragment of SEQ ID No. 2 has a length of at least 1000 nucleotides, preferably of at least 1050 or 1100 nucleotides, more preferably of at least 1150, 1200 or 1250 nucleotides and most preferably of at least 1300, 1350 or 1400 nucleotides. The fragment of SEQ ID No. 3 has a length of at least 1000, 1050, 1100 or 1150 nucleotides, preferably of at least 1200, 1250 or 1300 nucleotides, more preferably of at least 1350, 1400 or 1450 nucleotides and most preferably of at least 1500, 1550 or 1600 nucleotides.

The present invention further relates to the use of nucleic acid sequences which are at least 70%, 75% or 80% identical, preferably at least 81, 82, 83, 84, 85 or 86% identical, more preferably at least 87, 88, 89 or 90% identical, even more preferably at least 91, 92, 93, 94 or 95% identical and most preferably at least 96, 97, 98, 99 or 100% identical to the complete sequence according to any of SEQ ID Nos. 1, 3 or 5 or a fragment of any of these sequences and which encode a protein having the activity of a fructose dehydrogenase when expressed with the other two subunits of fructose dehydrogenase as specified above.

Within the meaning of the present invention, “sequence identity” denotes the degree of conformity with regard to the 5′-3′ sequence within a nucleic acid molecule in comparison to another nucleic acid molecule. Preferably, the “percentage of sequence identity” is calculated by comparing two optimally aligned sequences over a particular region, determining the number of positions at which the identical base or amino acid is present in both sequences in order to yield the number of matched positions, dividing the number of those matched positions by the total number of positions in the segment being compared and multiplying the result by 100. The sequence identity may be determined using a series of programs, which are based on various algorithms, such as BLASTN, ScanProsite, the laser gene software, etc. As an alternative, the BLAST program package of the National Center for Biotechnology Information (http://www.ncbi.nlm.nih.gov/) may be used applying the default parameters. Here, in addition, the program Sequencher (Gene Codes Corp., Ann Arbor, Mich., USA) using the “dirtydata”-algorithm for sequence comparisons was employed.

The sequence identity refers to the degree of the sequence identity over a length of at least 300 nucleotides, preferably of 320, 340, 360, 380, 400 or 420 nucleotides and more preferably of 440, 460, 480, 500, 520 or 540 nucleotides and most preferably the whole length of SEQ ID No. 1. The sequence identity refers to the degree of the sequence identity over a length of 1000 nucleotides, preferably of 1050, 1100, 1150 or 1200 nucleotides, more preferably of 1250, 1300, 1350 or 1400 nucleotides and most preferably over the whole length of SEQ ID No. 3. The sequence identity refers to the degree of the sequence identity over a length of 1000, 1050, 1100, 1150, 1200 or 1250 nucleotides, preferably of 1300, 1350, 1400 or 1450 nucleotides, more preferably of 1500, 1550 or 1600 nucleotides and most preferably the whole length of SEQ ID No. 5.

The nucleic acid sequence hybridizing under stringent conditions with a complementary sequence of a nucleic acid sequence according to any of SEQ ID Nos. 1, 3 or 5 or a fragment thereof encodes a protein having the activity of a fructose dehydrogenase when expressed with the other two subunits of fructose dehydrogenase as specified above.

The variants of the sequences according to SEQ ID Nos. 1, 3 and 5 have essentially the same activity as the fructose dehydrogenase subunits characterized by SEQ ID Nos. 1, 3 and 5, i.e. the activity of the variants when used in combination in a heterotrimeric complex is at least 50% or 60%, preferably at least 70% or 80%, more preferably 85% or 90% and most preferably at least 95% or 98% of the activity of the fructose dehydrogenase characterized by the sequences according to SEQ ID Nos. 1, 3 and 5 when used in combination in a heterotrimeric complex.

The activity of the fructose dehydrogenase can be compared by incubating the fructose dehdrogenase (composed of the three subunits as defined above) with a suitable acceptor of the hydrogen atoms and measuring the amount of 5-ketofructose produced.

The amount of 5-ketofructose which is produced by the action of a fructose dehydrogenase, either for determining the activity of a variant or for determining the amount of 5-ketofructose produced by the methods of the present invention, can be determined by HPLC or by an enzymatic assay using a 5-ketofructose reductase, for example from Tatumella morbirosei. The 5-ketofructose reductase catalyzes the conversion of 5-ketofructose to fructose using NADPH as hydrogen donor. The increase of NADP⁺ can then be measured photometrically at 340 nm.

In the method of the present invention the acetic acid bacterium is incubated with a solution comprising a carbohydrate. The solution comprising a carbohydrate may be a solution which only comprises the carbohydrate dissolved in a suitable solvent such as water or it may be a culture medium which contains nutrients and salts in addition to said carbohydrate.

The term “carbohydrate” is intended to comprise biological molecules which are composed of carbon, oxygen and hydrogen atoms and includes monosaccharides, disaccharides, oligosaccharides and polysaccharides. If the carbohydrate is fructose, it may be used by the acetic acid bacterium used in the method to produce 5-ketofructose directly as the substrate for 5-ketofructose synthesis. Alternatively, at least a part of the carbohydrate may be enzymatically converted to fructose which then serves as the substrate for 5-ketofructose synthesis. The carbohydrate is preferably selected from the group consisting of fructose, glucose, sucrose and starch. If the acetic acid bacterium is genetically modified to express a fructose dehydrogenase and does not contain any additional genetic modifications affecting carbohydrate metabolism, the carbohydrate is fructose. If the acetic acid bacterium is genetically modified to express fructose dehydrogenase and glucose isomerase, the carbohydrate is glucose. If the acetic acid bacterium is genetically modified to express fructose dehydrogenase and invertase, possibly together with glucose isomerase, the carbohydrate is sucrose. If the acetic acid bacterium is genetically modified to express fructose dehydrogenase, glucose isomerase, amylase and pullulanase, the carbohydrate is starch.

The step of isolating the 5-ketofructose after it has been produced in the acetic acid bacterium means that the 5-ketofructose is separated from the acetic acid bacteria producing it and other compounds which may be present in the solution of 5-ketofructose, such as medium components. Typically, the 5-ketofructose is first separated from the acetic acid bacterium by centrifugation or filtration and is then isolated by any known method including adsorption on activated carbon or other suitable adsorbers and treatment with cation or anion exchange resins. Suitable methods for isolating 5-ketofructose are described for example in U.S. Pat. No. 3,206,375 and in Avigad and Englard (1965) J. Biol. Chem. 240(6): 2290-2296.

In one embodiment of the present invention, the method for producing 5-ketofructose comprises the steps of:

(a) incubating a purified first acetic acid bacterium which is genetically modified to express a fructose dehydrogenase with a solution comprising a carbohydrate; and

(b) isolating the 5-ketofructose.

If fructose is used as the carbohydrate which serves as a substrate for the fructose dehydrogenase, the concentration of fructose in the solution which is incubated with the purified first acetic acid bacterium is 30 to 500 g/l, preferably 80 to 800 g/l or 100 to 600 g/l, more preferably 150 to 500 g/l or 180 to 400 g/l and most preferably 180 to 250 g/l. These concentrations are used, if said purified first acetic acid bacterium is incubated with the solution comprising fructose in batch mode, i.e. no fructose is added, after said purified first acetic acid bacterium has been contacted with said solution at the beginning of the process.

The incubation of said purified first acetic acid bacterium with said solution comprising a carbohydrate can be performed in fed-batch mode. This means that the incubation is started with a certain amount of said solution comprising a carbohydrate and at one or more later time-points of the process fresh solution comprising said carbohydrate is added. Hence, new substrate is available for the fructose dehydrogenase after the feeding. If a fructose feed is used in the fed-batch method as the carbohydrate which serves as a substrate for the fructose dehydrogenase, the concentration of fructose in the solution which is fed to the bacterial cells in the fed-batch mode is 300 to 1,500 g/l, or 500 to 1,300 g/l, or 700 to 1,200 g/l or 1,000 g/l.

The term “purified acetic acid bacterium” is intended to mean that the acetic acid bacterium has been separated from the culture medium, before it is incubated with the carbohydrate. Typically, the acetic acid bacterium is separated from the culture medium by centrifugation, optionally washed with a suitable wash solution such as 0.9% sodium chloride solution or a buffer solution and then resuspended in the solution comprising the carbohydrate, before it is incubated with said solution. The incubation time depends on the cell density and the concentration of the substrate and can easily be determined by the skilled person. Optionally, the purified acetic acid bacterium can be fed with additional solution comprising the carbohydrate after it has been incubated in the initial solution comprising the carbohydrate for a period of time.

In one embodiment of the present invention, the method for producing 5-ketofructose comprises the steps of:

(a) culturing a first acetic acid bacterium which is genetically modified to express a fructose dehydrogenase in a culture medium comprising a carbohydrate in fed-batch mode; and

(b) isolating the 5-ketofructose.

The terms “cell culture” and “culturing of cells” refer to the maintenance and propagation of cells, preferably bacterial cells and more preferably of acetic acid bacteria.

The terms “medium”, “cell culture medium” and “culture medium” are interchangeably used herein and refer to a solution containing nutrients which are required for growing bacterial cells. Typically, a cell culture medium for bacterial cells provides essential and non-essential amino acids, vitamins, energy sources such as a carbohydrate, and salts required by the cell for growth and/or survival. Most of the above components of the medium may be provided by adding yeast extract to the culture medium, thereby providing free amino acids, proteins, vitamins and nucleotides. The culture medium may further comprise a selection agent such as an antibiotic to select for the cells which have been genetically modified by the expression of an antibiotic resistance gene. If the introduced fructose dehydrogenase is expressed under the control of an inducible promoter, the medium may also comprise an inducing agent.

For culturing the bacterial cells different strategies are available, including batch culture, perfusion culture, continuous culture and fed-batch culture. Within the method of the present invention, preferably a fed-batch culture process is used. The terms “fed-batch culture process”, “under fed-batch conditions”, “extended batch” and “fed-batch mode” are used interchangeably herein. In fed-batch culture the culturing process is started with a certain volume of the basal medium and one or more feeds are fed at later time-point(s) of the culture process while no product is removed from the cell culture broth.

In the fed-batch mode used in the present invention the fed-batch culturing is preferably performed under non-limiting conditions. The term “non-limiting conditions” means that the carbohydrate which provides the substrate for the fructose dehydrogenase is not limited at any time during the feeding. Accordingly, the concentration of said carbohydrate is high enough to saturate the fructose dehydrogenase with fructose. Typically, the concentration of the carbohydrate is at least five-fold higher than the Monod constant for this carbohydrate. The carbohydrate which provides the substrate for the fructose dehydrogenase may be fructose which can be used as a substrate directly, or it may be glucose, sucrose or starch which has to be converted to fructose by the action of one or more enzymes. Preferably, the carbohydrate which provides the substrate for the fructose dehydrogenase is fructose. Accordingly, preferably the culture is not limited with respect to fructose.

At the beginning of the culture or during the culture additional cells may be added. Further, a method with cell retention or cell reuse may be used, for example in sequential batches or repeated fed-batch methods.

The term “basal medium” is intended to refer to the medium which is used from the beginning of the cell culture process. The bacterial cells are inoculated into the basal medium and grown in this medium for a certain period until the feeding is started. The basal medium meets the definition of the culture medium as provided above. In the methods of the present invention the cells are grown in the basal medium for a period of 10 to 24 hours, preferably of 12 to 22 hours, more preferably of 15 to 20 hours and most preferably of 18 hours.

The pH of the medium may be adjusted to a pH of 5 to 6 using NaOH, KOH, Ca(OH)₂, CaCO₃, (NH₄)OH or Mg(OH)₂ to avoid the acidification of the medium during fermentation. To avoid that the pH of the medium becomes alkaline, the pH may also be adjusted with HCl, H₂SO₄ or H₃PO₄.

To further increase the yield of 5-ketofructose, the cells may be cultured under phosphate limitation to limit the growth of the bacterial cells and to redirect the available carbon to product synthesis. The cells may also be cultured under nitrogen, potassium, magnesium or sulfur limitation.

The feed is added to the cell culture after the cells have been cultured in the basal medium for a certain period. Within the methods of the present invention the feed is preferably a solution of the carbohydrate which is to be used as a substrate for the production of the 5-ketofructose, for example a solution of the carbohydrate in water or a cell culture medium comprising said carbohydrate. The feed may be added continuously or as a bolus at defined time points. Preferably, the feed is added continuously. In the methods of the present invention, the feed is added for a period of 20 to 50 hours, preferably of 23 to 40 hours, more preferably for 25 to 35 hours and most preferably of 30 hours. After the feeding has been stopped, the cells may be kept in culture until all the fructose in the cell culture medium has been consumed, for example for another 10 to 40 hours, preferably for another 14 to 35 hours, more preferably for another 20 to 30 hours and most preferably for another 24 hours.

If fructose is added to the culture medium as the carbohydrate which serves as a substrate for the fructose dehydrogenase, the concentration of fructose in the culture medium is 30 to 300 g/l, preferably 50 to 250 g/l or 70 to 220 g/l, more preferably 100 to 200 g/l and most preferably 130 to 190 g/l. If the cells are cultured in fed-batch mode, the concentration of the fructose in the culture medium as listed above is the concentration of fructose when the cells are cultured in batch mode, before the fructose is fed.

If a fructose feed is used in the fed-batch method as the carbohydrate which serves as a substrate for the fructose dehydrogenase, the concentration of fructose in the solution which is fed to the bacterial cells in the fed-batch mode is 300 to 1,500 g/l or 500 to 1,300 g/l or 700 to 1,200 g/l or 1,000 g/l.

Hence, in one embodiment the method for producing 5-ketofructose comprises the steps of:
(a) culturing a first acetic acid bacterium which is genetically modified to express a fructose dehydrogenase in a basal medium comprising 50 to 250 g/l fructose;
(b) feeding the first acetic acid bacterium with a solution comprising 300 to 1,500 g/l fructose and
(c) isolating the 5-ketofructose.

Using the above method, the product yield, defined as the ratio of the amount of 5-ketofructose produced to the amount of fructose added to the cells, is at least 80% or 82%, preferably at least 84% or 86%, more preferably at least 88% and most preferably at least 90%.

Using the above method, a product titer of at least 300 g/l, preferably of at least 320 g/l or 340 g/l, more preferably of at least 360 g/l or 380 g/l and most preferably of at least 400 g/l is obtained.

Alternatively to adding a solution comprising the carbohydrate to the first acetic acid bacterium, the carbohydrate may also be added as a solid or suspension.

Hence, the present invention also relates to a method for producing 5-ketofructose in an acetic acid bacterium, comprising the steps of:

a) providing a first acetic acid bacterium which is genetically modified to express a fructose dehydrogenase in a solution or a cell culture medium;

b) adding a carbohydrate in solid form or as a suspension to the culture of the first acetic acid bacterium; and

c) isolating the 5-ketofructose.

Preferably, the carbohydrate is fructose. Also preferably, the first acetic acid bacterium is Gluconobacter oxydans.

The term “solid form” is intended to refer to any pourable material including, but not being limited to, crystals, agglomerates, granules, pills or tablets.

The term “suspension” is intended to refer to a composition comprising the carbohydrate and a solvent, wherein the amount of the carbohydrate in the solvent exceeds the solubility of the carbohydrate in said solvent so that the composition comprises both dissolved carbohydrate and solid carbohydrate. The solid carbohydrate may be dissolved after the suspension has been added to the cell culture medium.

The acetic acid bacterium may also use glucose as carbohydrate to produce 5-ketofructose. In this case, the glucose has to be converted to fructose by a glucose isomerase and the fructose is then used as a substrate for the fructose dehydrogenase. The glucose isomerase can be added as an isolated enzyme to the solution comprising glucose. Alternatively, the first acetic acid bacterium may be genetically modified to express the glucose isomerase. For example, the first acetic acid bacterium may comprise a second expression vector comprising a nucleic acid sequence which encodes a glucose isomerase or the nucleic acid sequences encoding the glucose isomerase and the fructose dehydrogenase may both be present on the first expression vector. In a third alternative a second acetic acid bacterium which is genetically modified to express a glucose isomerase, preferably comprises an expression vector comprising a nucleic acid sequence encoding the glucose isomerase, may be used in a mixed culture with the first acetic acid bacterium.

Nucleic acid sequences encoding suitable glucose isomerases which can be used in the methods of the present invention are known to the skilled person and include nucleic acid sequences from Bacillus coagulans (UniProt entry AJO024439.1), E. coli K12 (UniProt entry b3565) and Streptomyces griseus (NCBI entry SGR_RS05195) and Bifidobacterium adolescentis (encoding the protein with NCBI entry WP_085379355).

Since the acetic acid bacterium may metabolize the glucose also to gluconate by the action of a glucose dehydrogenase, the acetic acid bacteria used together with glucose as the carbohydrate for the production of 5-ketofructose should lack activity of a membrane-bound and preferably of both a membrane-bound and a soluble glucose dehydrogenase. If the acetic acid bacterium is Gluconobacter oxydans, the membrane-bound glucose dehydrogenase is encoded by a nucleic acid sequence selected from the group consisting of:
a) a nucleic acid sequence comprising the sequence according to SEQ ID No. 9 or a fragment thereof;
b) a nucleic acid sequence encoding a protein comprising the amino acid sequence according to SEQ ID No. 10;
c) a nucleic acid sequence comprising a sequence which is at least 70% identical to the sequence according to SEQ ID No. 9 or a fragment thereof;
d) a nucleic acid sequence hybridizing under stringent conditions with a complementary sequence of a nucleic acid sequence according to SEQ ID No. 9 or a fragment thereof; and
e) a nucleic acid sequence encoding the same membrane-bound glucose dehydrogenase as any of the nucleic acid sequences of (a) to (d) above, but differing from the nucleic acid sequences of (a) to (d) above due to the degeneracy of the genetic code.

The fragment of SEQ ID No. 9 has a length of at least 1,200 nucleotides, preferably of at least 1,300, 1,400 or 1,500 nucleotides, more preferably of at least 1,600, 1,700 or 1,800 nucleotides and most preferably of at least 1,900, 2,000, 2,100, 2,200, 2,300 or 2,400 nucleotides.

The sequence identity refers to the degree of the sequence identity over a length of at least 1,200 nucleotides, preferably of 1,300, 1,400, 1,500, 1,600, 1,700 or 1,800 nucleotides and more preferably of 1,900, 2,000, 2,100, 2,200, 2,300 or 2,400 nucleotides and most preferably the whole length of SEQ ID No. 9.

If the acetic acid bacterium is Gluconobacter oxydans, the soluble glucose dehydrogenase is encoded by a nucleic acid sequence selected from the group consisting of:
a) a nucleic acid sequence comprising the sequence according to SEQ ID No. 11 or a fragment thereof;
b) a nucleic acid sequence encoding a protein comprising the amino acid sequence according to SEQ ID No. 12;
c) a nucleic acid sequence comprising a sequence which is at least 70% identical to the sequence according to SEQ ID No. 11 or a fragment thereof;
d) a nucleic acid sequence hybridizing under stringent conditions with a complementary sequence of a nucleic acid sequence according to SEQ ID No. 11 or a fragment thereof; and
e) a nucleic acid sequence encoding the same soluble glucose dehydrogenase as any of the nucleic acid sequences of (a) to (d) above, but differing from the nucleic acid sequences of (a) to (d) above due to the degeneracy of the genetic code.

The fragment of SEQ ID No. 11 has a length of at least 400 nucleotides, preferably of at least 450, 500 or 550 nucleotides, more preferably of at least 600, 650 or 700 nucleotides and most preferably of at least 720, 740, 760, 770, 780 or 790 nucleotides.

The sequence identity refers to the degree of the sequence identity over a length of at least 400 nucleotides, preferably of 450, 500, 550, 600, 650 or 700 nucleotides and more preferably of 720, 740, 760, 770, 780 or 790 nucleotides and most preferably the whole length of SEQ ID No. 11.

Methods for providing acetic acid bacteria lacking the expression of one or more proteins are described in the prior art. For example, the article of Krajewski et al. (2010) Appl. Environm. Microbiol. 76(13): 4369-4376 describes the construction of mutant Gluconobacter oxydans strains lacking expression of a membrane-bound glucose dehydrogenase and/or a soluble glucose dehydrogenase.

Accordingly, in one embodiment the method for producing 5-ketofructose comprises the steps of:
(a) contacting a first acetic acid bacterium which is genetically modified to express a fructose dehydrogenase and which lacks expression of a membrane-bound glucose dehydrogenase with a solution comprising glucose and an isolated glucose isomerase in fed-batch mode; and
(b) isolating the 5-ketofructose.

Isolated glucose isomerase enzyme is commercially available, for example from Sigma Aldrich.

In one embodiment the method for producing 5-ketofructose comprises the steps of:
(a) contacting a first acetic acid bacterium which is genetically modified to express a fructose dehydrogenase and a glucose isomerase and which lacks expression of a membrane-bound glucose dehydrogenase with a solution comprising glucose in fed-batch mode; and
(b) isolating the 5-ketofructose.

In one embodiment the method for producing 5-ketofructose comprises the steps of:
(a) contacting a first acetic acid bacterium which is genetically modified to express a fructose dehydrogenase and which lacks expression of a membrane-bound glucose dehydrogenase and a second acetic acid bacterium which is genetically modified to express a glucose isomerase and which lacks expression of a membrane-bound glucose dehydrogenase with a solution comprising glucose in fed-batch mode; and
(b) isolating the 5-ketofructose.

In one embodiment of the present invention, the method for producing 5-ketofructose comprises the steps of:
(a) incubating a purified first acetic acid bacterium which is genetically modified to express a fructose dehydrogenase and a glucose isomerase and which lacks expression of a membrane-bound glucose dehydrogenase with a solution comprising glucose in fed-batch mode; and
(b) isolating the 5-ketofructose.

In one embodiment of the present invention, the method for producing 5-ketofructose comprises the steps of:
(a) incubating a purified first acetic acid bacterium which is genetically modified to express a fructose dehydrogenase and which lacks expression of a membrane-bound glucose dehydrogenase and a purified second acetic acid bacterium which is genetically modified to express a glucose isomerase and which lacks expression of a membrane-bound glucose dehydrogenase with a solution comprising glucose in fed-batch mode; and
(b) isolating the 5-ketofructose.

In one embodiment of the present invention, the method for producing 5-ketofructose comprises the steps of:
(a) culturing a first acetic acid bacterium which is genetically modified to express a fructose dehydrogenase and a glucose isomerase and which lacks expression of a membrane-bound glucose dehydrogenase in a culture medium comprising glucose in fed-batch mode; and
(b) isolating the 5-ketofructose.

The first acetic acid bacterium may comprise only one expression vector which comprises the nucleic acid sequences encoding both the fructose dehydrogenase and the glucose isomerase. Alternatively, the first acetic acid bacterium may comprise a first expression vector comprising a nucleic acid sequence encoding the fructose dehydrogenase and a second expression vector comprising a nucleic acid sequence encoding the glucose isomerase.

In one embodiment of the present invention, the method for producing 5-ketofructose comprises the steps of:
(a) culturing a first acetic acid bacterium which is genetically modified to express a fructose dehydrogenase and which lacks expression of a membrane-bound glucose dehydrogenase and a second acetic acid bacterium which is genetically modified to express a glucose isomerase and which lacks expression of a membrane-bound glucose dehydrogenase in a culture medium comprising glucose in fed-batch mode; and
(b) isolating the 5-ketofructose.

The acetic acid bacterium may also use sucrose as carbohydrate to produce 5-ketofructose. In this case, the sucrose has first to be cleaved into fructose and glucose by the action of an invertase and the fructose is then used as a substrate for the fructose dehydrogenase. The invertase can be added as an isolated enzyme to the solution comprising sucrose. Alternatively, the first acetic acid bacterium may be genetically modified to express the invertase. For example, it may comprise a second expression vector comprising a nucleic acid sequence which encodes an invertase or the nucleic acid sequences encoding the invertase and the fructose dehydrogenase may be present on the same expression vector. In a third alternative a second acetic acid bacterium may be used which is genetically modified to express an invertase, preferably comprises an expression vector comprising a nucleic acid sequence encoding the invertase.

The invertase may be encoded by a nucleic acid sequence selected from the group consisting of:
a) a nucleic acid sequence comprising the sequence according to SEQ ID No. 13 or a fragment thereof;
b) a nucleic acid sequence encoding a protein comprising the amino acid sequence according to SEQ ID No. 14;
c) a nucleic acid sequence comprising a sequence which is at least 70% identical to the sequence according to SEQ ID No. 13 or a fragment thereof;
d) a nucleic acid sequence hybridizing under stringent conditions with a complementary sequence of a nucleic acid sequence according to SEQ ID No. 13 or a fragment thereof; and
e) a nucleic acid sequence encoding the same invertase as any of the nucleic acid sequences of (a) to (d) above, but differing from the nucleic acid sequences of (a) to (d) above due to the degeneracy of the genetic code.

The fragment of SEQ ID No. 13 has a length of at least 600 nucleotides, preferably of at least 650, 700 or 750 nucleotides, more preferably of at least 800, 850 or 900 nucleotides and most preferably of at least 950, 1,000, 1,050, 1,100 or 1,150 nucleotides.

The sequence identity refers to the degree of the sequence identity over a length of at least 600 nucleotides, preferably of 650, 700, 750, 800, 850 or 900 nucleotides and more preferably of 950, 1,000, 1,050, 1,100 or 1,150 nucleotides and most preferably the whole length of SEQ ID No. 13.

The variants of the sequence according to SEQ ID No. 13 as defined above encode a protein which has essentially the same enzymatic activity as the invertase encoded by the nucleic acid sequence according to SEQ ID No. 13, i.e. the activity of the variants is at least 50% or 60%, preferably at least 70% or 80%, more preferably at least 85% or 90% and most preferably at least 95% or 98% of the invertase encoded by the nucleic acid sequence according to SEQ ID No. 13.

Preferably, the glucose obtained by cleaving the sucrose by the action of the invertase is converted to fructose by the action of a glucose isomerase to increase the amount of fructose which can be used as a substrate for the fructose dehydrogenase. The glucose isomerase can be added as an isolated enzyme to the solution comprising sucrose. Alternatively, the first acetic acid bacterium may be genetically modified to express the glucose isomerase. For example, it may comprise a second expression vector comprising a nucleic acid sequence which encodes an invertase and a third expression vector comprising a nucleic acid sequence which encodes a glucose isomerase or the nucleic acid sequences encoding the invertase, the glucose isomerase and the fructose dehydrogenase may be present on the same expression vector. In a third alternative a second acetic acid bacterium may be used which is genetically modified to express an invertase, preferably comprises an expression vector comprising a nucleic acid sequence encoding the invertase and a third acetic acid bacterium may be used which is genetically modified to express a glucose isomerase, preferably comprises an expression vector comprising a nucleic acid sequence encoding the glucose isomerase.

The skilled person knows that any combinations of the above approaches may be used. Any of these combinations are is also within the scope of the present invention. For example, the first acetic acid bacterium may be genetically modified to express a fructose dehydrogenase and a glucose isomerase and may be contacted with the solution comprising sucrose together with a second acetic acid bacterium which is genetically modified to express an invertase.

Since the acetic acid bacterium may metabolize the glucose also to gluconate by the action of a glucose dehydrogenase, any of the acetic acid bacteria used together with sucrose as the carbohydrate for the production of 5-ketofructose and expressing a glucose isomerase should lack activity of a membrane-bound glucose dehydrogenase. Preferably, the acetic acid bacterium lacks expression of both a membrane-bound and a soluble glucose dehydrogenase. Nucleic acid sequences encoding a membrane-bound and a soluble glucose dehydrogenase have been described above.

Accordingly, in one embodiment the method for producing 5-ketofructose comprises the steps of:
(a) contacting a first acetic acid bacterium which is genetically modified to express a fructose dehydrogenase and which lacks expression of a membrane-bound glucose dehydrogenase with a solution comprising sucrose, an isolated glucose isomerase and an isolated invertase in fed-batch mode; and
(b) isolating the 5-ketofructose.

In one embodiment the method for producing 5-ketofructose comprises the steps of:
(a) contacting a first acetic acid bacterium which is genetically modified to express a fructose dehydrogenase and a glucose isomerase and which lacks expression of a membrane-bound glucose dehydrogenase with a solution comprising sucrose and an isolated invertase in fed-batch mode; and
(b) isolating the 5-ketofructose

In one embodiment the method for producing 5-ketofructose comprises the steps of:
(a) contacting a first acetic acid bacterium which is genetically modified to express a fructose dehydrogenase and an invertase and which lacks expression of a membrane-bound glucose dehydrogenase with a solution comprising sucrose and an isolated glucose isomerase in fed-batch mode; and
(b) isolating the 5-ketofructose.

In one embodiment the method for producing 5-ketofructose comprises the steps of:
(a) contacting a first acetic acid bacterium which is genetically modified to express a fructose dehydrogenase, an invertase and a glucose isomerase and which lacks expression of a membrane-bound glucose dehydrogenase with a solution comprising sucrose in fed-batch mode; and
(b) isolating the 5-ketofructose.

In one embodiment the method for producing 5-ketofructose comprises the steps of:
(a) contacting a first acetic acid bacterium which is genetically modified to express a fructose dehydrogenase and which lacks expression of a membrane-bound glucose dehydrogenase, a second acetic acid bacterium which is genetically modified to express a glucose isomerase and which lacks expression of a membrane-bound glucose dehydrogenase and a third acetic acid bacterium which is genetically modified to express an invertase and which lacks expression of a membrane-bound glucose dehydrogenase with a solution comprising sucrose in fed-batch mode; and
(b) isolating the 5-ketofructose.

In one embodiment the method for producing 5-ketofructose comprises the steps of:
(a) contacting a first acetic acid bacterium which is genetically modified to express a fructose dehydrogenase and a glucose isomerase and which lacks expression of a membrane-bound glucose dehydrogenase and a second acetic acid bacterium which is genetically modified to express an invertase and which lacks expression of a membrane-bound glucose dehydrogenase with a solution comprising sucrose in fed-batch mode; and
(b) isolating the 5-ketofructose.

In one embodiment the method for producing 5-ketofructose comprises the steps of:
(a) contacting a first acetic acid bacterium which is genetically modified to express a fructose dehydrogenase and an invertase and which lacks expression of a membrane-bound glucose dehydrogenase and a second acetic acid bacterium which is genetically modified to express a glucose isomerase and which lacks expression of a membrane-bound glucose dehydrogenase with a solution comprising sucrose in fed-batch mode; and
(b) isolating the 5-ketofructose.

In one embodiment of the present invention, the method for producing 5-ketofructose comprises the steps of:
(a) incubating a purified first acetic acid bacterium which is genetically modified to express a fructose dehydrogenase, an invertase and a glucose isomerase and which lacks expression of a membrane-bound glucose dehydrogenase with a solution comprising sucrose in fed-batch mode; and
(b) isolating the 5-ketofructose.

In one embodiment of the present invention, the method for producing 5-ketofructose comprises the steps of:
(a) incubating a purified first acetic acid bacterium which is genetically modified to express a fructose dehydrogenase and which lacks expression of a membrane-bound glucose dehydrogenase, a purified second acetic acid bacterium which is genetically modified to express a glucose isomerase and which lacks expression of a membrane-bound glucose dehydrogenase and a purified third acetic acid bacterium which is genetically modified to express an invertase and which lacks expression of a membrane-bound glucose dehydrogenase with a solution comprising sucrose in fed-batch mode; and
(b) isolating the 5-ketofructose.

In one embodiment of the present invention, the method for producing 5-ketofructose comprises the steps of:
(a) culturing a first acetic acid bacterium which is genetically modified to express a fructose dehydrogenase, an invertase and a glucose isomerase and which lacks expression of a membrane-bound glucose dehydrogenase in a culture medium comprising sucrose in fed-batch mode; and
(b) isolating the 5-ketofructose.

In one embodiment of the present invention, the method for producing 5-ketofructose comprises the steps of:
(a) culturing a first acetic acid bacterium which is genetically modified to express a fructose dehydrogenase and an invertase and which lacks expression of a membrane-bound glucose dehydrogenase and a second acetic acid bacterium which is genetically modified to express a glucose isomerase and which lacks expression of a membrane-bound glucose dehydrogenase in a culture medium comprising sucrose in fed-batch mode; and
(b) isolating the 5-ketofructose.

In one embodiment of the present invention, the method for producing 5-ketofructose comprises the steps of:
(a) culturing a first acetic acid bacterium which is genetically modified to express a fructose dehydrogenase and a glucose isomerase and which lacks expression of a membrane-bound glucose dehydrogenase and a second acetic acid bacterium which is genetically modified to express an invertase and which lacks expression of a membrane-bound glucose dehydrogenase in a culture medium comprising sucrose in fed-batch mode; and
(b) isolating the 5-ketofructose.

In one embodiment of the present invention, the method for producing 5-ketofructose comprises the steps of:
(a) culturing a first acetic acid bacterium which is genetically modified to express a fructose dehydrogenase and which lacks expression of a membrane-bound glucose dehydrogenase and a second acetic acid bacterium which is genetically modified to express an invertase and a glucose isomerase and which lacks expression of a membrane-bound glucose dehydrogenase in a culture medium comprising sucrose in fed-batch mode; and
(b) isolating the 5-ketofructose.

In one embodiment of the present invention, the method for producing 5-ketofructose comprises the steps of:
(a) culturing a first acetic acid bacterium which is genetically modified to express a fructose dehydrogenase and which lacks expression of a membrane-bound glucose dehydrogenase, a second acetic acid bacterium which is genetically modified to express a glucose isomerase and which lacks expression of a membrane-bound glucose dehydrogenase and a third acetic acid bacterium which is genetically modified to express an invertase and which lacks expression of a membrane-bound glucose dehydrogenase in a culture medium comprising sucrose in fed-batch mode; and
(b) isolating the 5-ketofructose.

The concentration of sucrose in the culture medium may be 10 to 250 g/l, preferably 30 to 220 g/l, more preferably 50 to 200 g/l and most preferably 100 to 150 g/l.

As discussed above, the bacterium is cultured in the fed-batch mode wherein a solution comprising sucrose is fed to the cells after the cells have been cultured in basal medium comprising sucrose for a certain period of time. Preferably, the fed-batch culture with feeding of sucrose does not result in a limitation of fructose available to the fructose dehydrogenase.

The acetic acid bacterium may also use starch as carbohydrate to produce 5-ketofructose. In this case, the starch has first to be cleaved into glucose by an amylase and/or a glucoamylase and a pullulanase. The amylase cleaves the α-1,4 bonds within the starch and the pullulanase cleaves the α-1,6 bonds within the starch. The glucose is then converted to fructose by a glucose isomerase and the fructose is used as a substrate for the fructose dehydrogenase to produce 5-ketofructose.

The amylase, pullulanase and/or glucose isomerase can be added as isolated enzymes to the solution comprising starch. Alternatively, the first acetic acid bacterium may be genetically modified to express the amylase, pullulanase and/or glucose isomerase. For example, it may comprise a second expression vector comprising a nucleic acid sequence which encodes an amylase, a third expression vector comprising a nucleic acid sequence which encodes a pullulanase and a fourth expression vector comprising a nucleic acid sequence which encodes a glucose isomerase or the nucleic acid sequences encoding the amylase, the pullulanase, the glucose isomerase and the fructose dehydrogenase may be present on the first expression vector. In a third alternative a second acetic acid bacterium which is genetically modified to express an amylase, preferably comprises an expression vector comprising a nucleic acid sequence encoding the amylase, a third acetic acid bacterium which is genetically modified to express a pullulanase, preferably comprises an expression vector comprising a nucleic acid sequence encoding the pullulanase and/or a fourth acetic acid bacterium which is genetically modified to express a glucose isomerase, preferably comprises an expression vector comprising a nucleic acid sequence encoding the glucose isomerase, may be used.

The skilled person knows that any combinations of the above approaches may be used. Any of these combinations are is also within the scope of the present invention.

For example, the first acetic acid bacterium may be genetically modified to express a fructose dehydrogenase and a glucose isomerase and may be contacted with the solution comprising starch together with a second acetic acid bacterium which is genetically modified to express an amylase and a pullulanase.

Since the acetic acid bacterium may metabolize the glucose also to gluconate by the action of a glucose dehydrogenase, any of the acetic acid bacteria used together with starch as the carbohydrate for the production of 5-ketofructose should lack expression of a membrane-bound glucose dehydrogenase. Preferably, the acetic acid bacterium lacks expression of both a membrane-bound and a soluble glucose dehydrogenase. Nucleic acid sequences encoding a membrane-bound and a soluble glucose dehydrogenase have been described above.

Amylases and glucoamylases which can be used in the methods of the present invention are known to the skilled person and include the α-amylase from Streptomyces griseus (Cadenas et al. (1996) FEMS Microbiology Letters 137: 63-68), from Streptococcus bovis (Inokuma et al. (2015) Appl. Microbiol. Biotechnol. 99:1655-1663) or from Geobacillus stearothermophilus (Suzuki et al (2009) Appl. Microbiol. Biotechnol. 82:491-500) and the glucoamylase from Rhizopus oryzae (Inokuma et al. (2015) Appl. Microbiol. Biotechnol. 99:1655-1663)

Pullulanases which can be used in the methods of the present invention are known to the skilled person and include the pullulanase from Klebsiella pneumoniae (d'Enfert et al. (1987) EMBO J. 6(11):3531-3538), from Geobacillus thermoleovorans (Ayadi et al. (2008) Appl. Microbiol. Biotechnol. 78:473-481, from Bacillus naganoensis (Nie et al. (2013) PLoS ONE 8(10): e78416) and from Bacillus acidopullulyticus (Chen et al. (2014) J. Indust. Microbiol. Biotechnol. 41:1803-1810).

Accordingly, in one embodiment the method for producing 5-ketofructose comprises the steps of:
(a) contacting a first acetic acid bacterium which is genetically modified to express a fructose dehydrogenase and which lacks expression of a membrane-bound glucose dehydrogenase with a solution comprising starch, an isolated glucose isomerase, an isolated pullulanase and an isolated amylase in fed-batch mode; and
(b) isolating the 5-ketofructose

In one embodiment the method for producing 5-ketofructose comprises the steps of:
(a) contacting a first acetic acid bacterium which is genetically modified to express a fructose dehydrogenase and a glucose isomerase and which lacks expression of a membrane-bound glucose dehydrogenase with a solution comprising starch, an isolated amylase and an isolated pullulanase in fed-batch mode; and
(b) isolating the 5-ketofructose

In one embodiment the method for producing 5-ketofructose comprises the steps of:
(a) contacting a first acetic acid bacterium which is genetically modified to express a fructose dehydrogenase and an amylase and which lacks expression of a membrane-bound glucose dehydrogenase with a solution comprising starch, an isolated glucose isomerase and an isolated pullulanase in fed-batch mode; and
(b) isolating the 5-ketofructose.

In one embodiment the method for producing 5-ketofructose comprises the steps of:
(a) contacting a first acetic acid bacterium which is genetically modified to express a fructose dehydrogenase and a pullulanase and which lacks expression of a membrane-bound glucose dehydrogenase with a solution comprising starch, an isolated glucose isomerase and an isolated amylase in fed-batch mode; and
(b) isolating the 5-ketofructose.

Isolated amylases and pullulanases are available for example from Sigma Aldrich.

In one embodiment the method for producing 5-ketofructose comprises the steps of:
(a) contacting a first acetic acid bacterium which is genetically modified to express a fructose dehydrogenase, an amylase, a pullulanase and a glucose isomerase and which lacks expression of a membrane-bound glucose dehydrogenase with a solution comprising starch in fed-batch mode; and
(b) isolating the 5-ketofructose.

In one embodiment the method for producing 5-ketofructose comprises the steps of:
(a) contacting a first acetic acid bacterium which is genetically modified to express a fructose dehydrogenase and which lacks expression of a membrane-bound glucose dehydrogenase, a second acetic acid bacterium which is genetically modified to express a glucose isomerase and which lacks expression of a membrane-bound glucose dehydrogenase, a third acetic acid bacterium which is genetically modified to express an amylase and which lacks expression of a membrane-bound glucose dehydrogenase and a fourth acetic acid bacterium which is genetically modified to express a pullulanase with a solution comprising starch in fed-batch mode; and
(b) isolating the 5-ketofructose.

In one embodiment the method for producing 5-ketofructose comprises the steps of:
(a) contacting a first acetic acid bacterium which is genetically modified to express a fructose dehydrogenase and a glucose isomerase and which lacks expression of a membrane-bound glucose dehydrogenase and a second acetic acid bacterium which is genetically modified to express an amylase and a pullulanase and which lacks expression of a membrane-bound glucose dehydrogenase with a solution comprising starch in fed-batch mode; and
(b) isolating the 5-ketofructose.

In one embodiment the method for producing 5-ketofructose comprises the steps of:
(a) contacting a first acetic acid bacterium which is genetically modified to express a fructose dehydrogenase and an amylase and which lacks expression of a membrane-bound glucose dehydrogenase and a second acetic acid bacterium which is genetically modified to express a glucose isomerase and a pullulanase and which lacks expression of a membrane-bound glucose dehydrogenase with a solution comprising starch in fed-batch mode; and
(b) isolating the 5-ketofructose.

In one embodiment of the present invention, the method for producing 5-ketofructose comprises the steps of:
(a) incubating a purified first acetic acid bacterium which is genetically modified to express a fructose dehydrogenase, an amylase, a pullulanase and a glucose isomerase and which lacks expression of a membrane-bound glucose dehydrogenase with a solution comprising starch in fed-batch mode; and
(b) isolating the 5-ketofructose.

In one embodiment of the present invention, the method for producing 5-ketofructose comprises the steps of:
(a) incubating a purified first acetic acid bacterium which is genetically modified to express a fructose dehydrogenase and which lacks expression of a membrane-bound glucose dehydrogenase, a purified second acetic acid bacterium which is genetically modified to express a glucose isomerase and which lacks expression of a membrane-bound glucose dehydrogenase, a purified third acetic acid bacterium which is genetically modified to express an amylase and which lacks expression of a membrane-bound glucose dehydrogenase and a fourth acetic acid bacterium which is genetically modified to express a pullulanase with a solution comprising starch in fed-batch mode; and
(b) isolating the 5-ketofructose.

In one embodiment of the present invention, the method for producing 5-ketofructose comprises the steps of:
(a) culturing a first acetic acid bacterium which is genetically modified to express a fructose dehydrogenase, an amylase, a pullulanase and a glucose isomerase and which lacks expression of a membrane-bound glucose dehydrogenase in a culture medium comprising starch in fed-batch mode; and
(b) isolating the 5-ketofructose.

In one embodiment of the present invention, the method for producing 5-ketofructose comprises the steps of:
(a) culturing a first acetic acid bacterium which is genetically modified to express a fructose dehydrogenase and which lacks expression of a membrane-bound glucose dehydrogenase, a second acetic acid bacterium which is genetically modified to express a glucose isomerase and which lacks expression of a membrane-bound glucose dehydrogenase, a third acetic acid bacterium which is genetically modified to express an amylase and which lacks expression of a membrane-bound glucose dehydrogenase and a fourth acetic acid bacterium which is genetically modified to express a pullulanase and which lacks expression of a membrane-bound glucose dehydrogenase in a culture medium comprising starch in fed-batch mode; and
(b) isolating the 5-ketofructose.

As discussed above, the bacterium is cultured in the fed-batch mode wherein a solution comprising starch is fed to the cells after the cells have been cultured in basal medium comprising glucose, fructose or starch for a certain period of time. Preferably, the fed-batch culture with feeding of starch does not result in a limitation of fructose.

The method for producing 5-ketofructose may comprise the steps of:
(a) contacting a first acetic acid bacterium which is genetically modified to express a fructose dehydrogenase and which lacks expression of a membrane-bound glucose dehydrogenase with a solution comprising glucose and an isolated glucose isomerase; and
(b) isolating the 5-ketofructose.

The method for producing 5-ketofructose may comprise the steps of:
(a) contacting a first acetic acid bacterium which is genetically modified to express a fructose dehydrogenase and a glucose isomerase and which lacks expression of a membrane-bound glucose dehydrogenase with a solution comprising glucose; and
(b) isolating the 5-ketofructose.

The method for producing 5-ketofructose may comprise the steps of:
(a) contacting a first acetic acid bacterium which is genetically modified to express a fructose dehydrogenase and which lacks expression of a membrane-bound glucose dehydrogenase and a second acetic acid bacterium which is genetically modified to express a glucose isomerase and which lacks expression of a membrane-bound glucose dehydrogenase with a solution comprising glucose; and
(b) isolating the 5-ketofructose.

The method for producing 5-ketofructose may comprise the steps of:
(a) incubating a purified first acetic acid bacterium which is genetically modified to express a fructose dehydrogenase and a glucose isomerase and which lacks expression of a membrane-bound glucose dehydrogenase with a solution comprising glucose; and
(b) isolating the 5-ketofructose.

The method for producing 5-ketofructose may comprise the steps of:
(a) incubating a purified first acetic acid bacterium which is genetically modified to express a fructose dehydrogenase and which lacks expression of a membrane-bound glucose dehydrogenase and a purified second acetic acid bacterium which is genetically modified to express a glucose isomerase and which lacks expression of a membrane-bound glucose dehydrogenase with a solution comprising glucose; and
(b) isolating the 5-ketofructose.

The method for producing 5-ketofructose may comprise the steps of:
(a) culturing a first acetic acid bacterium which is genetically modified to express a fructose dehydrogenase and a glucose isomerase and which lacks expression of a membrane-bound glucose dehydrogenase in a culture medium comprising glucose; and
(b) isolating the 5-ketofructose.

The method for producing 5-ketofructose may comprise the steps of:
(a) contacting a first acetic acid bacterium which is genetically modified to express a fructose dehydrogenase and which lacks expression of a membrane-bound glucose dehydrogenase with a solution comprising sucrose, an isolated glucose isomerase and an isolated invertase; and
(b) isolating the 5-ketofructose.

The method for producing 5-ketofructose may comprise the steps of:
(a) contacting a first acetic acid bacterium which is genetically modified to express a fructose dehydrogenase and a glucose isomerase and which lacks expression of a membrane-bound glucose dehydrogenase with a solution comprising sucrose and an isolated invertase; and
(b) isolating the 5-ketofructose

The method for producing 5-ketofructose may comprise the steps of:
(a) contacting a first acetic acid bacterium which is genetically modified to express a fructose dehydrogenase and an invertase and which lacks expression of a membrane-bound glucose dehydrogenase with a solution comprising sucrose and an isolated glucose isomerase; and
(b) isolating the 5-ketofructose.

The method for producing 5-ketofructose may comprise the steps of:
(a) contacting a first acetic acid bacterium which is genetically modified to express a fructose dehydrogenase, an invertase and a glucose isomerase and which lacks expression of a membrane-bound glucose dehydrogenase with a solution comprising sucrose; and
(b) isolating the 5-ketofructose.

The method for producing 5-ketofructose may comprise the steps of:
(a) contacting a first acetic acid bacterium which is genetically modified to express a fructose dehydrogenase and which lacks expression of a membrane-bound glucose dehydrogenase, a second acetic acid bacterium which is genetically modified to express a glucose isomerase and which lacks expression of a membrane-bound glucose dehydrogenase and a third acetic acid bacterium which is genetically modified to express an invertase and which lacks expression of a membrane-bound glucose dehydrogenase with a solution comprising sucrose; and
(b) isolating the 5-ketofructose.

The method for producing 5-ketofructose may comprise the steps of:
(a) contacting a first acetic acid bacterium which is genetically modified to express a fructose dehydrogenase and a glucose isomerase and which lacks expression of a membrane-bound glucose dehydrogenase and a second acetic acid bacterium which is genetically modified to express an invertase and which lacks expression of a membrane-bound glucose dehydrogenase with a solution comprising sucrose; and
(b) isolating the 5-ketofructose.

The method for producing 5-ketofructose may comprise the steps of:
(a) contacting a first acetic acid bacterium which is genetically modified to express a fructose dehydrogenase and an invertase and which lacks expression of a membrane-bound glucose dehydrogenase and a second acetic acid bacterium which is genetically modified to express a glucose isomerase and which lacks expression of a membrane-bound glucose dehydrogenase with a solution comprising sucrose; and
(b) isolating the 5-ketofructose.

The method for producing 5-ketofructose may comprise the steps of:
(a) incubating a purified first acetic acid bacterium which is genetically modified to express a fructose dehydrogenase, an invertase and a glucose isomerase and which lacks expression of a membrane-bound glucose dehydrogenase with a solution comprising sucrose; and
(b) isolating the 5-ketofructose.

The method for producing 5-ketofructose may comprise the steps of:
(a) incubating a purified first acetic acid bacterium which is genetically modified to express a fructose dehydrogenase and which lacks expression of a membrane-bound glucose dehydrogenase, a purified second acetic acid bacterium which is genetically modified to express a glucose isomerase and which lacks expression of a membrane-bound glucose dehydrogenase and a purified third acetic acid bacterium which is genetically modified to express an invertase and which lacks expression of a membrane-bound glucose dehydrogenase with a solution comprising sucrose; and
(b) isolating the 5-ketofructose.

The method for producing 5-ketofructose may comprise the steps of:
(a) culturing a first acetic acid bacterium which is genetically modified to express a fructose dehydrogenase, an invertase and a glucose isomerase and which lacks expression of a membrane-bound glucose dehydrogenase in a culture medium comprising sucrose; and
(b) isolating the 5-ketofructose.

The method for producing 5-ketofructose may comprise the steps of:
(a) culturing a first acetic acid bacterium which is genetically modified to express a fructose dehydrogenase and an invertase and which lacks expression of a membrane-bound glucose dehydrogenase and a second acetic acid bacterium which is genetically modified to express a glucose isomerase and which lacks expression of a membrane-bound glucose dehydrogenase in a culture medium comprising sucrose; and
(b) isolating the 5-ketofructose.

The method for producing 5-ketofructose may comprise the steps of:
(a) culturing a first acetic acid bacterium which is genetically modified to express a fructose dehydrogenase and a glucose isomerase and which lacks expression of a membrane-bound glucose dehydrogenase and a second acetic acid bacterium which is genetically modified to express an invertase and which lacks expression of a membrane-bound glucose dehydrogenase in a culture medium comprising sucrose; and
(b) isolating the 5-ketofructose.

The method for producing 5-ketofructose may comprise the steps of:
(a) culturing a first acetic acid bacterium which is genetically modified to express a fructose dehydrogenase and which lacks expression of a membrane-bound glucose dehydrogenase and a second acetic acid bacterium which is genetically modified to express an invertase and a glucose isomerase and which lacks expression of a membrane-bound glucose dehydrogenase in a culture medium comprising sucrose; and
(b) isolating the 5-ketofructose.

The method for producing 5-ketofructose may comprise the steps of:
(a) culturing a first acetic acid bacterium which is genetically modified to express a fructose dehydrogenase and which lacks expression of a membrane-bound glucose dehydrogenase, a second acetic acid bacterium which is genetically modified to express a glucose isomerase and which lacks expression of a membrane-bound glucose dehydrogenase and a third acetic acid bacterium which is genetically modified to express an invertase and which lacks expression of a membrane-bound glucose dehydrogenase in a culture medium comprising sucrose; and
(b) isolating the 5-ketofructose.

The method for producing 5-ketofructose may comprise the steps of:
(a) contacting a first acetic acid bacterium which is genetically modified to express a fructose dehydrogenase and which lacks expression of a membrane-bound glucose dehydrogenase with a solution comprising starch, an isolated glucose isomerase, an isolated pullulanase and an isolated amylase; and
(b) isolating the 5-ketofructose

The method for producing 5-ketofructose may comprise the steps of:
(a) contacting a first acetic acid bacterium which is genetically modified to express a fructose dehydrogenase and a glucose isomerase and which lacks expression of a membrane-bound glucose dehydrogenase with a solution comprising starch, an isolated amylase and an isolated pullulanase; and
(b) isolating the 5-ketofructose

The method for producing 5-ketofructose may comprise the steps of:
(a) contacting a first acetic acid bacterium which is genetically modified to express a fructose dehydrogenase and an amylase and which lacks expression of a membrane-bound glucose dehydrogenase with a solution comprising starch, an isolated glucose isomerase and an isolated pullulanase; and
(b) isolating the 5-ketofructose.

The method for producing 5-ketofructose may comprise the steps of:
(a) contacting a first acetic acid bacterium which is genetically modified to express a fructose dehydrogenase and a pullulanase and which lacks expression of a membrane-bound glucose dehydrogenase with a solution comprising starch, an isolated glucose isomerase and an isolated amylase; and
(b) isolating the 5-ketofructose.

The method for producing 5-ketofructose may comprise the steps of:
(a) contacting a first acetic acid bacterium which is genetically modified to express a fructose dehydrogenase, an amylase, a pullulanase and a glucose isomerase and which lacks expression of a membrane-bound glucose dehydrogenase with a solution comprising starch; and
(b) isolating the 5-ketofructose.

The method for producing 5-ketofructose may comprise the steps of:
(a) contacting a first acetic acid bacterium which is genetically modified to express a fructose dehydrogenase and which lacks expression of a membrane-bound glucose dehydrogenase, a second acetic acid bacterium which is genetically modified to express a glucose isomerase and which lacks expression of a membrane-bound glucose dehydrogenase, a third acetic acid bacterium which is genetically modified to express an amylase and which lacks expression of a membrane-bound glucose dehydrogenase and a fourth acetic acid bacterium which is genetically modified to express a pullulanase with a solution comprising starch; and
(b) isolating the 5-ketofructose.

The method for producing 5-ketofructose may comprise the steps of:
(a) contacting a first acetic acid bacterium which is genetically modified to express a fructose dehydrogenase and a glucose isomerase and which lacks expression of a membrane-bound glucose dehydrogenase and a second acetic acid bacterium which is genetically modified to express an amylase and a pullulanase and which lacks expression of a membrane-bound glucose dehydrogenase with a solution comprising starch; and
(b) isolating the 5-ketofructose.

The method for producing 5-ketofructose may comprise the steps of:
(a) contacting a first acetic acid bacterium which is genetically modified to express a fructose dehydrogenase and an amylase and which lacks expression of a membrane-bound glucose dehydrogenase and a second acetic acid bacterium which is genetically modified to express a glucose isomerase and a pullulanase and which lacks expression of a membrane-bound glucose dehydrogenase with a solution comprising starch; and
(b) isolating the 5-ketofructose.

The method for producing 5-ketofructose may comprise the steps of:
(a) incubating a purified first acetic acid bacterium which is genetically modified to express a fructose dehydrogenase, an amylase, a pullulanase and a glucose isomerase and which lacks expression of a membrane-bound glucose dehydrogenase with a solution comprising starch; and
(b) isolating the 5-ketofructose.

The method for producing 5-ketofructose may comprise the steps of:
(a) incubating a purified first acetic acid bacterium which is genetically modified to express a fructose dehydrogenase and which lacks expression of a membrane-bound glucose dehydrogenase, a purified second acetic acid bacterium which is genetically modified to express a glucose isomerase and which lacks expression of a membrane-bound glucose dehydrogenase, a purified third acetic acid bacterium which is genetically modified to express an amylase and which lacks expression of a membrane-bound glucose dehydrogenase and a fourth acetic acid bacterium which is genetically modified to express a pullulanase with a solution comprising starch; and
(b) isolating the 5-ketofructose.

The method for producing 5-ketofructose may comprise the steps of:
(a) culturing a first acetic acid bacterium which is genetically modified to express a fructose dehydrogenase, an amylase, a pullulanase and a glucose isomerase and which lacks expression of a membrane-bound glucose dehydrogenase in a culture medium comprising starch; and
(b) isolating the 5-ketofructose.

The method for producing 5-ketofructose may comprise the steps of:
(a) culturing a first acetic acid bacterium which is genetically modified to express a fructose dehydrogenase and which lacks expression of a membrane-bound glucose dehydrogenase, a second acetic acid bacterium which is genetically modified to express a glucose isomerase and which lacks expression of a membrane-bound glucose dehydrogenase, a third acetic acid bacterium which is genetically modified to express an amylase and which lacks expression of a membrane-bound glucose dehydrogenase and a fourth acetic acid bacterium which is genetically modified to express a pullulanase and which lacks expression of a membrane-bound glucose dehydrogenase in a culture medium comprising starch; and
(b) isolating the 5-ketofructose.

The acetic acid bacterium may additionally comprise a genetic modification enabling the export of the proteins to the outside of the bacterial cell. The genetic modification may be such that it causes a leaky outer membrane. Genetic modifications causing a leaky outer membrane include the deletion of any of the genes selected from the group consisting of tolA, tolB, tolQRA, rfa, pal und lpp (see Lazzaroni et al. (1999) FEMS Microbiol. Lett 177: 191-197). Preferably, the genetic modification enabling the export of proteins and causing a leaky outer membrane is a deletion of the tolB gene.

The tolB gene may have a nucleic acid sequence selected from the group consisting of:
a) a nucleic acid sequence comprising the sequence according to SEQ ID No. 15 or a fragment thereof;
b) a nucleic acid sequence encoding a protein comprising the amino acid sequence according to SEQ ID No. 16;
c) a nucleic acid sequence comprising a sequence which is at least 70% identical to the sequence according to SEQ ID No. 15 or a fragment thereof;
d) a nucleic acid sequence hybridizing under stringent conditions with a complementary sequence of a nucleic acid sequence according to SEQ ID No. 15 or a fragment thereof; and
e) a nucleic acid sequence encoding the same TolB protein as any of the nucleic acid sequences of (a) to (d) above, but differing from the nucleic acid sequences of (a) to (d) above due to the degeneracy of the genetic code.

The fragment of SEQ ID No. 15 has a length of at least 650 nucleotides, preferably of at least 700, 750 or 800 nucleotides, more preferably of at least 850, 900 or 950 nucleotides and most preferably of at least 1,000, 1,050, 1,100 1,150, 1,200 or 1,250 nucleotides.

The sequence identity refers to the degree of the sequence identity over a length of at least 650 nucleotides, preferably of 700, 750, 800, 850, 900 or 950 nucleotides and more preferably of 1,000, 1,050, 1,100, 1,150, 1,200 or 1,250 nucleotides and most preferably the whole length of SEQ ID No. 15.

The article of Kosciow et al. (2016) J. Ind. Microbiol. Biotechnol. 43: 989-999 describes the construction of a mutant Gluconobacter oxydans strain with a deletion of the tolB gene.

The 5-ketofructose produced by any of the methods described above can be used to prepare a sweetener composition. A sweetener composition is a composition which is used to provide sweet taste to food such as desserts, fruit preparations, marmalade, chocolate and chocolate products, cakes, bread, joghurt, cereals, pudding, softdrinks, pickled vegetables, canned fruit and sauces. The sweetener composition can be added to the food before consumption or already during preparation of the food product.

The sweetener composition may be in any form which is suitable for the intended use. These forms include, but are not limited to, a solution, drops, gels, crystals, powder, granules and pills.

The sweetener composition may comprise only the 5-ketofructose and no further ingredients. Alternatively, the sweetener composition may comprise one or more additional compounds which are compatible with food, such as extenders, binders, stabilizers, preservers, flavourings and desiccants.

For oral consumption the 5-ketofructose may be mixed with acceptable excipients or carriers such as, but not being limited to, sodium hydrogen carbonate, lactose, tartaric acid, calcium carbonate, calcium phosphate, microcrystalline cellulose, dextrose, magnesium stearate, sucrose, plant gums, methylcellulose, povidone, carboxymethylcellulose and hydroxypropylmethylcellulose.

The 5-ketofructose produced by any of the methods described above can also be used to produce a non-alcoholic beverage comprising the 5-ketofructose. Non-alcoholic beverages typically include carbonated water, a sweetener and one or more natural or artificial flavourings, but do not include alcohol. They may further include caffeine, taurine, colorings and/or preservatives. Typical non-alcoholic beverages include, but are not limited to, coke, lemonade, mixtures of coke and lemonade, diluted and sweetened fruit juices, ice tea and energy drinks.

The present invention further relates to a non-alcoholic beverage containing 5-ketofructose.

The following examples and figures are provided for illustrative purposes. It is thus understood that the examples and figures are not to be construed as limiting. The skilled person in the art will clearly be able to envisage further modifications of the principles laid out herein.

### EXAMPLES

### Example 1: Comparison of 5-Ketofructose Production in Wild-Type Strain and Recombinant Strain Expressing Fructose Dehydrogenase

The Gluconobacter oxydans wild-type strain 621H ΔhsdR and a genetically modified Gluconobacter oxydans strain transformed with plasmid pBBR1p264-FDH Strep (see SEQ ID No. 17) were cultured in a medium comprising 6 g/l yeast extract, 100 mM fructose and 100 mM potassium phosphate buffer, pH 6.8 at a temperature of 30° C. for 72 hours. At different time-points samples were taken and the amount of fructose and 5-ketofructose was determined by HPLC. To this end, the samples were centrifuged, the supernatant was filtered and diluted with water containing 5 mM H₂SO₄. The samples were analyzed with a Knauer HPLC system using the column Aminex-HPX87H 300×7.8 mm (Bio-Rad) and the following conditions: mobile phase: 5 mM H₂SO₄, temperature 21° C. for fructose determination and 65° C. for 5-ketofructose determination, flow rate 0.3 ml/min, injection volume 20 μl. Detection was performed by using the Knauer UV-detector 2600 and RI-detector 101. For analysis the software ChromeGate Client (version 3.1.7) was used.

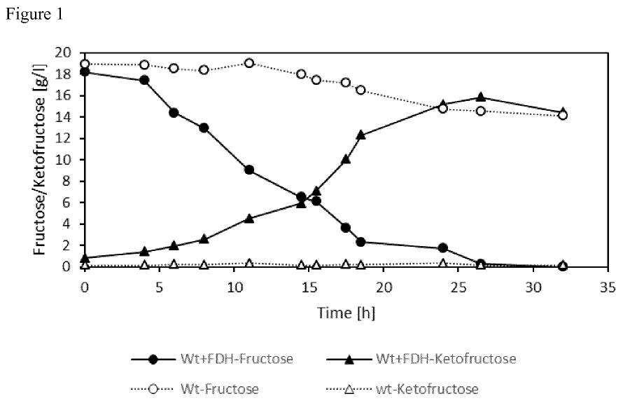

The results of this experiment are shown in FIG. 1. The wild-type strain does not convert the fructose to 5-ketofructose, but only to biomass. The conversion of fructose is slow so that after 32 hours only a quarter of the fructose is degraded. In contrast, the Glucose oxydans strain converts 18 g/l fructose to 16 g/l 5-ketofructose within 27 hours.

### Example 2: Production of 5-Ketofructose Using Fructose as Carbohydrate

The Gluconobacter oxydans strain transformed with plasmid pBBR1p264-FDH Strep (SEQ ID No. 17) was cultured in a complex medium comprising 150 g/l fructose, 5 g/l yeast extract, 1 g/l potassium dihydrogen phosphate, 1 g/l ammonium sulfate, 2.5 g/l magnesium-heptahydrate and 50 μg/ml kanamycin. For the preculture 80 g/l mannitol were used instead of fructose as the carbon source. The pH in the medium of the pre culture and the main culture was set to pH 6 and was not regulated.

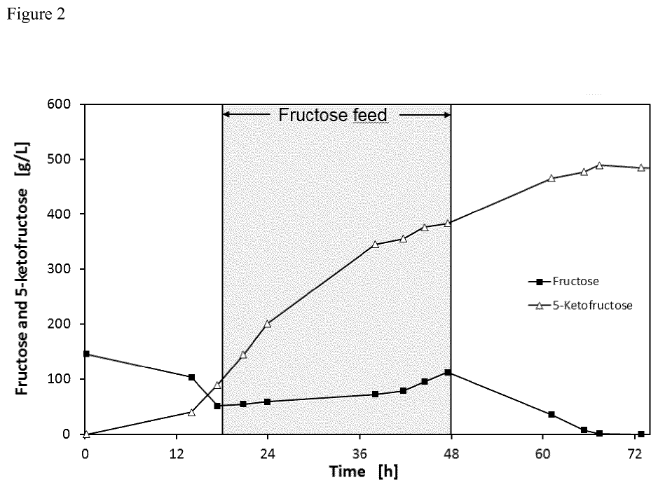

In the fed-batch phase a solution of 1,035 g/l fructose in water was fed and the pH was regulated to pH 5.5 with 3 mM KOH and 2 M HCl. In both the batch and the fed-batch phase the temperature was 30° C., the dissolved oxygen concentration pO₂ was ≥30% and the gassing was 1 L/min. In the batch phase the maximum stirrer speed was 1,308 rpm. In the fed-batch phase the cells were fed with 850 ml of a 1,035 g/l fructose solution at a feed rate of 26 ml/h and the maximum stirrer speed was 1,500 rpm. At different time-points samples were taken and the amount of fructose and 5-ketofructose was determined by HPLC as described in Example 1. The results of this experiment are shown in FIG. 2.

After 18 h in the batch phase a feed with 1,035 g/l fructose was started. After 48 hours the feed was stopped and the culture was continued for another 22 hours until the fructose was completely consumed. From the 562 g/l fructose introduced 489 g/l 5-ketofructose were obtained, which corresponds to a product yield of 87%.

### Example 3: Production of 5-Ketofructose Using Sucrose as Carbohydrate

For producing 5-ketofructose from sucrose a mixed culture of two genetically modified Gluconobacter oxydans strains was used. The first strain was Gluconobacter oxydans 621H Δhsd ΔtolB containing the plasmid pBBR1p264-sacC which expresses an invertase and has a deletion of the tolB gene leading to a leaky outer membrane (SEQ ID No. 18). The second strain was a genetically modified Gluconobacter oxydans strain transformed with plasmid pBBR1p264-FDH Strep and therefore overexpressing a fructose dehydrogenase.

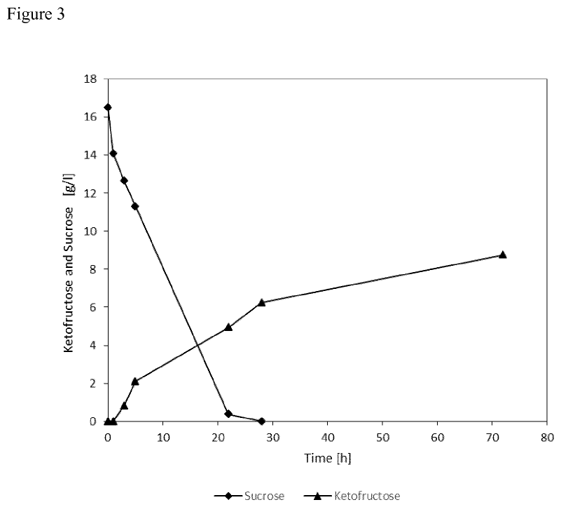

In a preculture on 9 g/l glucose both strains were cultured at 30° C. and 180 rpm to an OD₆₀₀ of 0.6. Then the two strains were combined and 17 g/l sucrose was added. At different time-points samples were taken and the amount of fructose and 5-ketofructose was determined by HPLC as described in Example 1. The results of this experiment are shown in FIG. 3.

In the mixed culture the fructose obtained by cleaving the sucrose was converted almost completely to 8.7 g/l 5-ketofructose. Since in this experiment the strains did not comprise a deletion of glucose dehydrogenase and since no glucose isomerase was used to convert glucose to fructose, the glucose from the preculture and from the cleavage of sucrose was converted to 2-ketogluconate via gluconate and to acetate.

Nevertheless, this example shows that sucrose can be used as a substrate for 5-ketofructose production.

### Example 4: Production of 5-Ketofructose Using Sucrose as Carbohydrate and a Single Gluconobacter Strain

The modified Gluconobacter oxydans strain contains a genome-encoded fructose dehydrogenase complex (fdhSLC) which was prepared by replacing the endogenous glucose dehydrogenase gene with the genes encoding the fructose dehydrogenase complex according to the method described in Kostner et al. (2013) Appl. Microbiol. Biotechnol. 97: 8341-8349. In addition, for the hydrolysis of sucrose the gene sacC, encoding an extracellular invertase from Zymomonas mobilis ZM4 (see SEQ ID No. 14), was cloned into a pBBR1MCS-based expression vector downstream of the strong constitutive promotor p264 (SEQ ID NO. 7).

The modified Gluconobacter strain was cultured in 100 ml culture medium containing yeast extract (0.6% w/v) and sucrose (final concentration of 100 mM) with cefoxitin (50 μg/ml). Kanamycin (50 μg/ml) was added for plasmid maintenance. The cultures were buffered with carbonate (final concentration of 100 mM) and incubated at 28° C. and 180 rpm for 52 hours.

The concentration of sucrose, fructose and 5-ketofructose was determined using HPLC. 1 ml samples were taken from cultures and centrifuged at 13,000×g for 1 min. Supernatants were diluted 1:10 with 5 mM H₂SO₄, and applied to an Aminex-HPX87H column using 5 mM H₂SO₄ as eluent. The concentration of sucrose and fructose was analysed at 25° C. with a flow rate of 0.6 ml min⁻¹, while 5-ketofructose was quantified at 65° C. Sugars were detected by refractive index and acids were detected by UV-VIS at 210 nm and quantified by comparison to calibration curves.

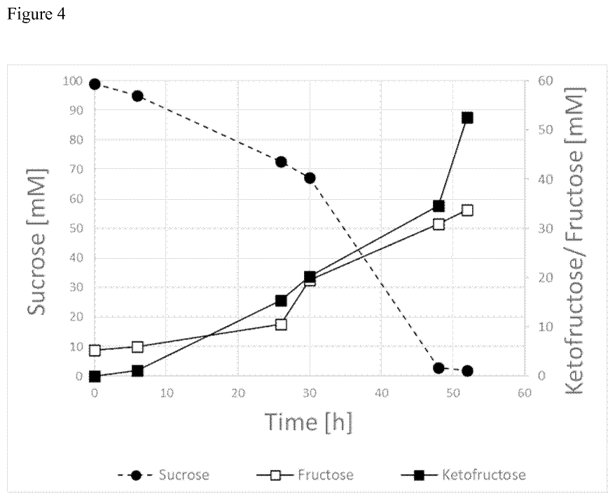

HPLC analysis showed that sucrose was completely hydrolyzed within 52 hours (see FIG. 4). The concentration of fructose increased from 5 to 30 mM within 52 hours. In addition, the production of 5-KF with a concentration up to 52 mM could be detected. In summary, half of the available fructose units from sucrose could be converted to 5-KF with the one-strain system.

1. A method for producing 5-ketofructose in an acetic acid bacterium, comprising the steps of:
a) contacting a first acetic acid bacterium which is genetically modified to express a fructose dehydrogenase with a solution comprising a carbohydrate in fed-batch mode; and
b) isolating the 5-ketofructose.

2. The method of claim 1, wherein the first acetic acid bacterium comprises a first expression vector comprising a nucleic acid sequence which encodes a fructose dehydrogenase.

3. The method of claim 1, wherein the acetic acid bacterium is from the genus Gluconobacter.

4.-6. (canceled)

7. The method of claim 1, wherein step (a) comprises incubating a purified acetic acid bacterium with a solution comprising said carbohydrate in fed-batch mode.

8. The method of claim 1, wherein step (a) comprises culturing the acetic acid bacterium in a culture medium comprising said carbohydrate in fed-batch mode.

9. (canceled)

10. The method of claim 1, wherein the carbohydrate is fructose.

11.-12. (canceled)

13. The method of claim 1, wherein the carbohydrate is glucose, and the glucose is converted to fructose by the action of a glucose isomerase, and wherein the acetic acid bacterium lacks expression of a membrane-bound glucose dehydrogenase.

14. The method of claim 13, wherein an isolated glucose isomerase is added to the solution comprising glucose.

15. The method of claim 13, wherein the first acetic acid bacterium is genetically modified to express a glucose isomerase.

16. (canceled)

17. The method of claim 13, wherein a second acetic acid bacterium genetically modified to express a glucose isomerase is contacted with said solution comprising glucose.

18. (canceled)

19. The method of claim 1, wherein the carbohydrate is sucrose and the sucrose is converted to fructose by the action of a glucose isomerase and an invertase, and wherein the acetic acid bacterium lacks expression of a membrane-bound glucose dehydrogenase.

20. The method of claim 19, wherein an isolated glucose isomerase and/or invertase is added to the solution comprising sucrose.

21. The method of claim 19, wherein the first acetic acid bacterium is genetically modified to express a glucose isomerase and/or an invertase.

22. (canceled)

23. The method of claim 19, wherein a second acetic acid bacterium genetically modified to express a glucose isomerase and/or an invertase is contacted with said solution comprising sucrose.

24.-26. (canceled)

27. The method of claim 1, wherein the carbohydrate is starch and the starch is converted to fructose by the action of an amylase, a pullulanase, and a glucose isomerase, and wherein the acetic acid bacterium lacks expression of a membrane-bound glucose dehydrogenase.

28. The method of claim 27, wherein an isolated glucose isomerase and/or amylase and/or pullulanase is added to the solution comprising starch.

29. The method of claim 27, wherein the first acetic acid bacterium is genetically modified to express a glucose isomerase, an amylase and/or a pullulanase.

30. (canceled)

31. The method of claim 27, wherein a second acetic acid bacterium genetically modified to express a glucose isomerase and/or a third acetic acid bacterium genetically modified to express an amylase and/or a fourth acetic acid bacterium genetically modified to express a pullulanase are/is contacted with said solution comprising starch.

32.-35. (canceled)

36. A method for producing a sweetener composition, comprising producing 5-ketofructose by the method of claim 1 and preparing a sweetener composition comprising 5-ketofructose.

37. The method of claim 36, wherein the sweetener composition is a powder, solution, granule or pill.

38. A method for producing a non-alcoholic beverage containing 5-ketofructose, comprising producing 5-ketofructose by the method of claim 1 and adding the 5-ketofructose to a non-alcoholic beverage.

39-59. (canceled)


SEQUENCE LISTING (18 sequences)

SEQ ID NO: 1 - DNA, 552 bp, Gluconobacter japonicus
atggaaaaaatagctgattccggccctgttcaaatctttctttcgcgtagaaagcttctggctttttccggtgcctgcctaacagtcgcagcaattggaacaccatcgaaaggatctactcaggacgttgtggcattaaatcgcgatagcatttcggattttatgcagctttctgcttttgccaccggtcacaaaaatctggatctcaatatcggatcagcacttctcttggcatttgaagctcagaagcatgatttttctgctcaaataaaggctctccgcgaacatattactaaaaataattatcaggatgttgaagcactggatgcagccatgaaagatgaccccctgcatcccacgctcctccagattatccgggcgtggtattcaggtgtcatcgaagacggaacaaacgctaaagtttacgctttcgaaaaagcactcatgtatcagccgtcccgcgatgtcgtggtcattccgacatatgctcataacgggcccaattactgggtctcggaacccgcatccgtcgatgtcatgccggcattttaa

SEQ ID NO: 2 - protein, 183 aa, Gluconobacter japonicus
MEKIADSGPVQIFLSRRKLLAFSGACLTVAAIGTPSKGSTQDVVALNRDSISDFMQLSAFATGHKNLDLNIGSALLLAFEAQKHDFSAQIKALREHITKNNYQDVEALDAAMKDDPLHPTLLQIIRAWYSGVIEDGTNAKVYAFEKALMYQPSRDVVVIPTYAHNGPNYWVSEPASVDVMPAF

SEQ ID NO: 3 - DNA, 1461 bp, Gluconobacter japonicus
atgcgatattttcgccctctgtccgccacagccatgacaaccgttctgcttctcgcaggcacgaacgtacgggcgcaaccgacagaaccaacacctgcttcagcccatagcccctccatcagccgcggtcattatctggcaattgccgccgattgtgcggcctgccataccaatgggcggaacggtcagttccttgctggcggctatgccatttcttccccaatgggaaatatttactcaaccaatattacgccctcgaagacgcacggtatcggcaactatactctggagcagttttctaaagcacttcggcatggtatccgcgccgatggcgctcaactgtatcccgccatgccttatgacgcttacaatcgtctgacggatgaagacgtcaaatcgctctacgcttacatcatgactgaagtaaaacccgtagatgcaccttctcccaagacgcagctccccttcccgttttcaatccgtgcatcactcggtatttggaaaattgcggcaaggatcgaaggcaaaccctatgtctttgatcatacgcacaatgacgactggaaccgtggtcgctatctggtcgatgagctagcccattgcggtgaatgtcacacaccgcgtaactttcttctggctcccaatcagtccgcttatcttgctggtgcggacataggatcatggcgtgcaccaaatattaccaatgaccctcaaagcggcattggcagttggtcggatcaggatctcttccaatatctgaagactggaaaaaccgcacatgctcgtgcagcaggcccaatggcagaagcaatcgagcacagcctacaatatcttccggatgccgatatttctgccataattacatatctgcgttcggttccggcaaaggcagaaagcggtcaaacagtcgctaattttgagcatgccggccgcccatccggttacagtgttgctgacgctaactcccgtcgcaccaacagtactctaacaaaaacaaccgatggtgctgctctctacgaggctgtgtgcgccagttgccatcaatctgacggtaaagggtccaaagacggttattatccgtccctcgtaggaaatacgacgaccgggcagctcaatccaaatgatctgatcgccagtattctttacggtgtggaccgcacaacggataaccacgaaatcctgatgccggcttttggtccagactctctcgtacagcccctgacagatgagcagatcgccacaatcgcggactatgttctaagccactttggcaatgctcaggccacagtctcggcagatgcggtcaaacaggtgcgggcgggcggaaagcaagttcctctcgccaggctcgccagtccgggcgtgatgctgcttctcggagctggtggcatcctgggagccattctggtggtaggcggcctctggtggctgatcagccgtcgcaaaaagcgttccgcttaa

SEQ ID NO: 4 - protein, 486 aa, Gluconobacter japonicus
MRYFRPLSATAMTTVLLLAGTNVRAQPTEPTPASAHSPSISRGHYLAIAADCAACHTNGRNGQFLAGGYAISSPMGNIYSTNITPSKTHGIGNYTLEQFSKALRHGIRADGAQLYPAMPYDAYNRLTDEDVKSLYAYIMTEVKPVDAPSPKTQLPFPFSIRASLGIWKIAARIEGKPYVFDHTHNDDWNRGRYLVDELAHCGECHTPRNFLLAPNQSAYLAGADIGSWRAPNITNDPQSGIGSWSDQDLFQYLKTGKTAHARAAGPMAEAIEHSLQYLPDADISAIITYLRSVPAKAESGQTVANFEHAGRPSGYSVADANSRRTNSTLTKTTDGAALYEAVCASCHQSDGKGSKDGYYPSLVGNTTTGQLNPNDLIASILYGVDRTTDNHEILMPAFGPDSLVQPLTDEQIATIADYVLSHFGNAQATVSADAVKQVRAGGKQVPLARLASPGVMLLLGAGGILGAILVVGGLWWLISRRKKRSA

SEQ ID NO: 5 - DNA, 1635 bp, Gluconobacter japonicus
atgtctaatgaaacgctttcggctgatgtcgtcatcattggagcaggaatctgcggctccctgctcgcccacaaactcgtccgcaacggactttctgtgctgctcctggatgcaggtccccgccgggaccgctcccagatcgtagaaaactggcgcaacatgccgcctgacaacaagtcccagtatgattatgcaacaccttaccccagcgtaccttgggccccgcataccaactacttcccagacaataactacctgattgttaaaggcccggaccggacggcttacaagcaaggaatcatcaaaggcgtcgggggtacaacctggcactgggcggcttcaagctggcggtacctgcccaacgatttcaagctgcattccacatatggcgtgggccgcgattacgccatgagttacgacgaacttgagccgtactattacgaggccgagtgcgaaatgggcgtgatgggaccaaacggcgaagagattacaccgtctgcgcctcgccaaaatccatggccgatgacctccatgccttacggatatggagaccggactttcacggagatcgtcagcaagctcggtttctcaaacactcctgttccgcaggcacggaacagccgtccttatgatggccgaccacaatgctgtggtaacaacaactgcatgccaatctgcccgattggcgcgatgtacaacggcgtatacgcggcaataaaagcggaaaagctgggcgcaaaaattatccccaatgctgtcgtctacgcgatggaaacggatgcaaaaaaccgcattacagcgattagcttctatgatcccgacaagcagtcccatcgcgtcgttgcaaaaacattcgtgatcgccgcaaacgggatcgaaacaccgaaactgctgcttctggcggcaaatgatcgaaaccctcatgggattgccaactcatcagaccttgttggccggaacatgatggaccatccgggcatcggcatgagcttccagtctgcggagcccatctgggctggtggaggctcagtccagatgagttccatcaccaacttccgtgatggcgatttccgctcggagtatgctgccacgcagatcggctacaacaatacggctcagaactcccgcgccggcatgaaagccctgtccatggggttggttggcaagaagctggatgaagaaatccgccgtcgcacggcgcatggtgtggacatttatgccaaccatgaagtcctcccggaccccaacaaccgtcttgttctctccaaagaccataaggatgcgctcggtattccacatcctgaagtcacctacgatgttggggagtatgttcgaaagtcagctgtcatctcacgacagcgcctgatggatatcgccaaagccatgggcggtacggaaatcgagatgactccgtattttacgcccaacaaccacatcaccggtggcactatcatgggccatgatccacgggattcagtggtcgataaatggctccggacccatgatcattccaatctgttccttgcaacaggcgccacaatggcagcgtccggtacggtcaattcaacgttaacaatggccgcactgtcattacgcgcggcagatgccattctcaatgacctgaaacaggggtaa

SEQ ID NO: 6 - protein, 544 aa, Gluconobacter japonicus
MSNETLSADVVIIGAGICGSLLAHKLVRNGLSVLLLDAGPRRDRSQIVENWRNMPPDNKSQYDYATPYPSVPWAPHTNYFPDNNYLIVKGPDRTAYKQGIIKGVGGTTWHWAASSWRYLPNDFKLHSTYGVGRDYAMSYDELEPYYYEAECEMGVMGPNGEEITPSAPRQNPWPMTSMPYGYGDRTFTEIVSKLGFSNTPVPQARNSRPYDGRPQCCGNNNCMPICPIGAMYNGVYAAIKAEKLGAKIIPNAVVYAMETDAKNRITAISFYDPDKQSHRVVAKTFVIAANGIETPKLLLLAANDRNPHGIANSSDLVGRNMMDHPGIGMSFQSAEPIWAGGGSVQMSSITNFRDGDFRSEYAATQIGYNNTAQNSRAGMKALSMGLVGKKLDEEIRRRTAHGVDIYANHEVLPDPNNRLVLSKDHKDALGIPHPEVTYDVGEYVRKSAVISRQRLMDIAKAMGGTEIEMTPYFTPNNHITGGTIMGHDPRDSVVDKWLRTHDHSNLFLATGATMAASGTVNSTLTMAALSLRAADAILNDLKQG

SEQ ID NO: 7 - DNA, 310 bp, Gluconobacter oxydans
gatgttcctggatctgttgttctgatgcatcacggtgacttccggacaaagcacatggatgggcgtcaagggtcaagatgtcagaaggttcatcttgctcacgtcactgtgaggactttcacgtcgtcgtgacttgaggaaaggaaggacttatgattgagcgttcaagaaaataatgcgcttgcaggccacttgggcttacgataaggacaccgcttccgcgcacgcgtccgggccacgagtggcatgcccggccgtgcagagtctctgcaggatgcaggaccaatcgtttacggcgagggagaccgaa

SEQ ID NO: 8 - DNA, 190 bp, Gluconobacter oxydans
gttgatgttcctgtctgaaacggccccgcacaggcgtcgggcgcgaacggtctgtgttgcggcaggataccattgtcgcgacatggggcaatgcagacgtcgtttttgttgcggtaactggataccgcatggctgaaaacggcggaaaaccgctgcgaatcacataattcaatgtgtgaacggggcttga

SEQ ID NO: 9 - DNA, 2427 bp, Gluconobacter oxydans
atgagcacaacatcccggccagggctctgggccctgattacggccgcggtattcgcgctttgcggcgcgatccttaccgttggcggcgcatgggtcgctgccatcggcggccctctctattatgtcatccttggcctggcacttctcgccacggctttcctctcattcaggcgcaacccggctgccctctacctgttcgcagtcgtcgtcttcggaacggtcatctgggaactcaccgttgtcggtctcgacatctgggccctgatcccgcgctcggacatcgtcatcatcctcggcatctggctgctgttgccgttcgtctcccgccagatcggcggcacgcggacgaccgtcctgccgctcgccggtgccgttggcgttgcggttctggcgctgttcgccagcctcttcaccgacccgcatgacatcagtggcgacctgccgacgcagatcgcaaacgcctcccccgccgacccggacaacgtcccggccagcgagtggcacgcctatggtcgtacgcaggccggtgaccgctggtccccgctgaaccagatcaacgcgtccaacgtcagcaacctcaaggtcgcatggcatatccacaccaaggatatgatgaactccaacgacccgggcgaagcgacgaacgaagcgaccccgatcgagttcaacaacacgctttatatgtgctcgctgcaccagaagctgtttgcggttgatggtgccaccggtaacgtcaagtgggtctacgatccgaagctccagatcaaccctggcttccagcatctgacctgccgtggcgtcagcttccacgaaacgccggccaatgccacggattccgatggcaatcctgctccgacggactgcgccaagcgcatcatcctgccggtcaatgatggccgtctggttgaagtcgatgccgacacgggcaagacctgctccggcttcggcaacaatggcgagatcgatctgcgcgttccgaaccagccttacacgacgcccggccagtacgagccgacgtccccgccggtcatcacggacaagctgatcatcgccaacagcgccatcaccgataacggttcggtcaagcaggcttcgggcgccacgcaggcattcgacgtctacaccggcaagcgtgtctgggtgttcgatgcgtccaacccggacccgaaccagcttccggatgacagccaccctgtcttccacccgaactccccgaactcctggatcgtgtcgtcttacgacaggaacctgaacctcgtgtacatcccgatgggcgtgggtactcccgatcagtggggcggtgaccgcacgaaggattccgagcgtttcgctccgggtatcgttgctctgaacgcagacacgggcaagctcgcctggttctaccagaccgttcatcacgatctgtgggacatggacgttccgtcacagccgagcctcgtggacgtaacgcagaaggacggcacgcttgttccggccatctacgctccgaccaagaccggcgacatcttcgtcctcgaccgtcgtaccggcaaggaaatcgtcccggctccggaaaccccggttccccagggcgccgctccgggcgatcacaccagcccgacccagccgatgtcgcagctgaccctgcgtccgaagaacccgctgaacgactccgatatctggggcggcacgatcttcgaccagatgttctgcagcatctatttccacaccctgcgctacgaaggccccttcacgccgccgtcgctcaagggctcgctcatcttcccgggcgatctgggaatgttcgagtggggtggtctggccgtcgatccgcagcgtcaggtggctttcgccaacccgatctccctgccgttcgtctcccagcttgttccccgtggaccgggcaacccgctctggcctgaaaaggacgccaagggtacgggtggtgaaaccggcctgcagcacaactatggcattccgtatgccgtcaacctgcatccgttcctggatccggtgctgctgccgttcggcatcaagatgccctgccgtacgccgccctggggctatgtcgccggtattgacctgaagaccaacaaggtcgtctggcagcaccgcaacggcaccctgcgtgactcgatgtatggcagctccctgccgatcccgctgccgccgatcaagatcggtgtcccgagcctcggtggcccgctctccacggctggcaacctcggcttcctgacggcgtccatggattactacatccgtgcgtacaacctgacgacgggcaaggtgctgtggcaggaccgtctgccggctggcgcccaggcaacgccgatcacctatgccatcaacggcaagcagtacatcgtgacctatgcaggcggacacaactcgttcccgacccgcatgggcgacgacatcatcgcctacgccctgcccgatcagaaatga

SEQ ID NO: 10 - protein, 808 aa, Gluconobacter oxydans
MSTTSRPGLWALITAAVFALCGAILTVGGAWVAAIGGPLYYVILGLALLATAFLSFRRNPAALYLFAVVVFGTVIWELTVVGLDIWALIPRSDIVIILGIWLLLPFVSRQIGGTRTTVLPLAGAVGVAVLALFASLFTDPHDISGDLPTQIANASPADPDNVPASEWHAYGRTQAGDRWSPLNQINASNVSNLKVAWHIHTKDMMNSNDPGEATNEATPIEFNNTLYMCSLHQKLFAVDGATGNVKWVYDPKLQINPGFQHLTCRGVSFHETPANATDSDGNPAPTDCAKRIILPVNDGRLVEVDADTGKTCSGFGNNGEIDLRVPNQPYTTPGQYEPTSPPVITDKLIIANSAITDNGSVKQASGATQAFDVYTGKRVWVFDASNPDPNQLPDDSHPVFHPNSPNSWIVSSYDRNLNLVYIPMGVGTPDQWGGDRTKDSERFAPGIVALNADTGKLAWFYQTVHHDLWDMDVPSQPSLVDVTQKDGTLVPAIYAPTKTGDIFVLDRRTGKEIVPAPETPVPQGAAPGDHTSPTQPMSQLTLRPKNPLNDSDIWGGTIFDQMFCSIYFHTLRYEGPFTPPSLKGSLIFPGDLGMFEWGGLAVDPQRQVAFANPISLPFVSQLVPRGPGNPLWPEKDAKGTGGETGLQHNYGIPYAVNLHPFLDPVLLPFGIKMPCRTPPWGYVAGIDLKTNKVVWQHRNGTLRDSMYGSSLPIPLPPIKIGVPSLGGPLSTAGNLGFLTASMDYYIRAYNLTTGKVLWQDRLPAGAQATPITYAINGKQYIVTYAGGHNSFPTRMGDDIIAYALPDQK

SEQ ID NO: 11 - DNA, 801 bp, Gluconobacter oxydans
atgcctgccccttacaaagaccgtttcgccggcaagaaagtcctcgtcaccggggcctcccagggaattggcgaggccaccgcgcttcgttttgccgaagaaggcgcgcaggtcgccctcaacggccgcaaggaagacaagctgatcgccgtccgcgagaagctgcccaaggtttccggcggagagcacccgatcgccacgggtgacatttccaaagaagacgacgtcaaacgtctggttgccgagagcatcaaggccatgggtggtctcgacgttctggtctgcaatgcgggctatcagatcccctccccgtcagaagacatcaagctcgaagattttgaaggcgtgatggccgtcaacgtcacgggggtgatgctgccctgtcgcgaagtcatccgctactggctggaaaacggcatcaagggcacgatcattgtgaactcctccgttcaccagatcatcccgaaaccgcattatctgggctattccgcctccaagggtgccgtcggcaacattgtccgcacgctggcactggaatatgccacccgcggcatccgggtgaatgccgtggcgcccggcgccatcgtgacgccgatcaacatgtcgtggatcgacgatcccgaacagtacaaggccgtttcgagccacatcccgatgaagcgcccgggcgaaagccgcgaaatcgcggatgccatcaccttcctcgccgccgaggacagcacctacatcacgggtcagaccctgtatgtcgatggtggtctgacgctctacggcgatttcgaaaacaactggtcctcgtaa

SEQ ID NO: 12 - protein, 266 aa, Gluconobacter oxydans
MPAPYKDRFAGKKVLVTGASQGIGEATALRFAEEGAQVALNGRKEDKLIAVREKLPKVSGGEHPIATGDISKEDDVKRLVAESIKAMGGLDVLVCNAGYQIPSPSEDIKLEDFEGVMAVNVTGVMLPCREVIRYWLENGIKGTIIVNSSVHQIIPKPHYLGYSASKGAVGNIVRTLALEYATRGIRVNAVAPGAIVTPINMSWIDDPEQYKAVSSHIPMKRPGESREIADAITFLAAEDSTYITGQTLYVDGGLTLYGDFENNWSS

SEQ ID NO: 13 - DNA, 1242 bp, Zymomonas mobilis
atgtttaattttaatgccagtcgctggacgcgagcccaagcgatgaaagtgaataaatttgatttgacgacctctatgccggaaatcggcactgattttcctattatgcgtgatgacttgtggctgtgggatacttggccattacgggatatcaatggcaatcctgtcagctttaaaggctggaatgttattttctctttggtcgctgaccgcaatattccgtggaatgatcgccattctcatgcccgcatcggctatttctattctaaagatggtaaaagctgggtttatggtggccatctattgcaagaattggccaatagccgcacggcagaatggtccggcggcacgattatggcaccgggttcccgtaatcaggtcgaaaccttctttacctcgactttattcgacaagaatggcgtcagagaagccgttgctgctgttacgaaaggccgcatttatgcggatagtgaaggcgtttggttcaagggttttgaccagtcaacggatttgtttcaggctgatggtctgttttatcaaaattatgcagaaaataatctctggaatttccgtgatcctcatgttttcatcaatcctgaggatggtgagacctatgctttgttcgaagccaatgttgccactgtccgtggagaagacgatataggcgaggatgaaattggccctgttccggccaatacggtcgttccaaaagatgccaatttatgttcggcctctattggtattgcgcgttgtttgtcgccggatcgcaccgaatgggagctgttaccgcctttgttgacagcctttggcgtcaatgaccagatggaacggcctcatgtcattttccagaatggtttgacctatctctttacgatcagccatgattcgacttatgccgatggcttaaccggttccgatggtctttatggctttgtttccgaaaatggtatttttggcccctatgagccgctgaatggttctggccttgttctcggtggccctgcgtcacagccgaccgaagcctatgctcattacatcatgaataatggcttggttgaatcttttatcaatgaaatcattgatcccaaaagcggcaaagtcattgcgggcggtagcttggcaccgacggttcgcgttgaattacagggacatgagactttcgcgaccgaagtctttgattatggatatattcccgcatcttatgcttggccggtatggcctttcccggatcgtcgcaaataa

SEQ ID NO: 14 - protein, 413 aa, Zymomonas mobilis
MFNFNASRWTRAQAMKVNKFDLTTSMPEIGTDFPIMRDDLWLWDTWPLRDINGNPVSFKGWNVIFSLVADRNIPWNDRHSHARIGYFYSKDGKSWVYGGHLLQELANSRTAEWSGGTIMAPGSRNQVETFFTSTLFDKNGVREAVAAVTKGRIYADSEGVWFKGFDQSTDLFQADGLFYQNYAENNLWNFRDPHVFINPEDGETYALFEANVATVRGEDDIGEDEIGPVPANTVVPKDANLCSASIGIARCLSPDRTEWELLPPLLTAFGVNDQMERPHVIFQNGLTYLFTISHDSTYADGLTGSDGLYGFVSENGIFGPYEPLNGSGLVLGGPASQPTEAYAHYIMNNGLVESFINEIIDPKSGKVIAGGSLAPTVRVELQGHETFATEVFDYGYIPASYAWPVWPFPDRRK

SEQ ID NO: 15 - DNA, 1362 bp, Gluconobacter oxydans
atgtccttcattcccaataccgaagccgaggccctgagcgcgctgttttcgcgccgctccgttctgggagcgaccgccgcgggcggtctcctggcgacgcctctggcggcctttgctcagtccggtgcggcctccggccccggtgaagcagaaatcaccgtcgatcaggcccgtcaggagccgattcccatcgttgttccgaatttcggggcagggctgggtgagcagatctccggcgtgatcacgtccgacctgaacggaaccggtctgttcaaggtcatgagcggcagtgtgccgcccggctctacgccggatttcagcgcgctcaaagcccagggcgcccgcgcggccgtggccggtcaggccgttggtgcgggaagtgtgcgcgtcgagatgcgtctgtgggatgtgctctccggacagcagcttcagggcacggcctatacggcgtccaacagcaactggcgccggattgcgcatatcattgctgacgtgatctatgagcggatgctgggtgagaagggctatttcgatacccgcatcgcctatatcgcccgcaccggtccgcgccatcaccagatcacgcgtctggccctgatggatcaggatggcgccaacgagcgcatgctgacgggtggcgagtggctgacgctgacgccgcgtttcaacccggtgcgcgatcagatcgcgttcatgtcctacgccaacaaccgtccgcgcgtgtatctgttcgatctggcttctggccggcagcagattctgggcgagttcgcgggcatttcctttgcgccgcgcttctcgccgacaggtggctcggtggtgctgtccgctacgcgcgggggcgggtctgacatcttcgttgtcgatctggcttcccgtgcgaagcgacagatcaccaattccaatggcgcgatcgacaccagcccgtgcttcagcccggatggtagccagatcgtcttcaactccgatcgtggtggcagcccccagctctacatcatgagtgcatccggtggggcggcgaagcggatttcctatggcagtggccagtatggatccccggtctggtccccgcgtggagatctgatcgcctttacccggattggcagcggcgggttctcgctcggtgtcatgaacccggatggtacgggcgagcgtatcctgacgcagggcttcacggttgatggggcgacgttctgtccgaacggtcgtgtgctggcgttctgccgccagagcgcgtccggtgcgggcggtgcgggttttgcgtccgggattggtacgatcgatattacgggcttcaacgagcgtccgatccggatctcgacgggggcgtccgacccggcctggtctccgcggaacggctga

SEQ ID NO: 16 - protein, 453 aa, Gluconobacter oxydans
MSFIPNTEAEALSALFSRRSVLGATAAGGLLATPLAAFAQSGAASGPGEAEITVDQARQEPIPIVVPNFGAGLGEQISGVITSDLNGTGLFKVMSGSVPPGSTPDFSALKAQGARAAVAGQAVGAGSVRVEMRLWDVLSGQQLQGTAYTASNSNWRRIAHIIADVIYERMLGEKGYFDTRIAYIARTGPRHHQITRLALMDQDGANERMLTGGEWLTLTPRFNPVRDQIAFMSYANNRPRVYLFDLASGRQQILGEFAGISFAPRFSPTGGSVVLSATRGGGSDIFVVDLASRAKRQITNSNGAIDTSPCFSPDGSQIVFNSDRGGSPQLYIMSASGGAAKRISYGSGQYGSPVWSPRGDLIAFTRIGSGGFSLGVMNPDGTGERILTQGFTVDGATFCPNGRVLAFCRQSASGAGGAGFASGIGTIDITGFNERPIRISTGASDPAWSPRNG

SEQ ID NO: 17 - DNA, 9326 bp, artificial
  plasmid pBBR1p264-fdh-strep
taatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggtctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctcgttgcgcctgaatgagaggaaagccgtggcgagaagtgccaggccaaggatgacataatagagagggccgccgatggcagcgacccatgcgccgccaacggtaaggatcgcgccgcaaagcgcgaataccgcggccgtaatcagggcccagagccctggccgggatgttgtgctcatgatgttcctggatctgttgttctgatgcatcacggtgacttccggacaaagcacatggatgggcgtcaagggtcaagatgtcagaaggttcatcttgctcacgtcactgtgaggactttcacgtcgtcgtgacttgaggaaaggaaggacttatgattgagcgttcaagaaaataatgcgcttgcaggccacttgggcttacgataaggacaccgcttccgcgcacgcgtccgggccacgagtggcatgcccggccgtgcagagtctctgcaggatgcaggaccaatcgtttacggcgagggagaccgaatttgggctgcaggaattcgatatcaagcttatcgattcaggagaaggccaatggaaaaaatagctgattccggccctgttcaaatctttctttcgcgtagaaagcttctggctttttccggtgcctgcctaacagtcgcagcaattggaacaccatcgaaaggatctactcaggacgttgtggcattaaatcgcgatagcatttcggattttatgcagctttctgcttttgccaccggtcacaaaaatctggatctcaatatcggatcagcacttctcttggcatttgaagctcagaagcatgatttttctgctcaaataaaggctctccgcgaacatattactaaaaataattatcaggatgttgaagcactggatgcagccatgaaagatgaccccctgcatcccacgctcctccagattatccgggcgtggtattcaggtgtcatcgaagacggaacaaacgctaaagtttacgctttcgaaaaagcactcatgtatcagccgtcccgcgatgtcgtggtcattccgacatatgctcataacgggcccaattactgggtctcggaacccgcatccgtcgatgtcatgccggcattttaaggaccttattccgtgattatgcgatattttcgccctctgtccgccacagccatgacaaccgttctgcttctcgcaggcacgaacgtacgggcgcaaccgacagaaccaacacctgcttcagcccatagcccctccatcagccgcggtcattatctggcaattgccgccgattgtgcggcctgccataccaatgggcggaacggtcagttccttgctggcggctatgccatttcttccccaatgggaaatatttactcaaccaatattacgccctcgaagacgcacggtatcggcaactatactctggagcagttttctaaagcacttcggcatggtatccgcgccgatggcgctcaactgtatcccgccatgccttatgacgcttacaatcgtctgacggatgaagacgtcaaatcgctctacgcttacatcatgactgaagtaaaacccgtagatgcaccttctcccaagacgcagctccccttcccgttttcaatccgtgcatcactcggtatttggaaaattgcggcaaggatcgaaggcaaaccctatgtctttgatcatacgcacaatgacgactggaaccgtggtcgctatctggtcgatgagctagcccattgcggtgaatgtcacacaccgcgtaactttcttctggctcccaatcagtccgcttatcttgctggtgcggacataggatcatggcgtgcaccaaatattaccaatgaccctcaaagcggcattggcagttggtcggatcaggatctcttccaatatctgaagactggaaaaaccgcacatgctcgtgcagcaggcccaatggcagaagcaatcgagcacagcctacaatatcttccggatgccgatatttctgccataattacatatctgcgttcggttccggcaaaggcagaaagcggtcaaacagtcgctaattttgagcatgccggccgcccatccggttacagtgttgctgacgctaactcccgtcgcaccaacagtactctaacaaaaacaaccgatggtgctgctctctacgaggctgtgtgcgccagttgccatcaatctgacggtaaagggtccaaagacggttattatccgtccctcgtaggaaatacgacgaccgggcagctcaatccaaatgatctgatcgccagtattctttacggtgtggaccgcacaacggataaccacgaaatcctgatgccggcttttggtccagactctctcgtacagcccctgacagatgagcagatcgccacaatcgcggactatgttctaagccactttggcaatgctcaggccacagtctcggcagatgcggtcaaacaggtgcgggcgggcggaaagcaagttcctctcgccaggctcgccagtccgggcgtgatgctgcttctcggagctggtggcatcctgggagccattctggtggtaggcggcctctggtggctgatcagccgtcgcaaaaagcgttccgcttaataagattcaaaggtcaaaatcatgtctaatgaaacgctttcggctgatgtcgtcatcattggagcaggaatctgcggctccctgctcgcccacaaactcgtccgcaacggactttctgtgctgctcctggatgcaggtccccgccgggaccgctcccagatcgtagaaaactggcgcaacatgccgcctgacaacaagtcccagtatgattatgcaacaccttaccccagcgtaccttgggccccgcataccaactacttcccagacaataactacctgattgttaaaggcccggaccggacggcttacaagcaaggaatcatcaaaggcgtcgggggtacaacctggcactgggcggcttcaagctggcggtacctgcccaacgatttcaagctgcattccacatatggcgtgggccgcgattacgccatgagttacgacgaacttgagccgtactattacgaggccgagtgcgaaatgggcgtgatgggaccaaacggcgaagagattacaccgtctgcgcctcgccaaaatccatggccgatgacctccatgccttacggatatggagaccggactttcacggagatcgtcagcaagctcggtttctcaaacactcctgttccgcaggcacggaacagccgtccttatgatggccgaccacaatgctgtggtaacaacaactgcatgccaatctgcccgattggcgcgatgtacaacggcgtatacgcggcaataaaagcggaaaagctgggcgcaaaaattatccccaatgctgtcgtctacgcgatggaaacggatgcaaaaaaccgcattacagcgattagcttctatgatcccgacaagcagtcccatcgcgtcgttgcaaaaacattcgtgatcgccgcaaacgggatcgaaacaccgaaactgctgcttctggcggcaaatgatcgaaaccctcatgggattgccaactcatcagaccttgttggccggaacatgatggaccatccgggcatcggcatgagcttccagtctgcggagcccatctgggctggtggaggctcagtccagatgagttccatcaccaacttccgtgatggcgatttccgctcggagtatgctgccacgcagatcggctacaacaatacggctcagaactcccgcgccggcatgaaagccctgtccatggggttggttggcaagaagctggatgaagaaatccgccgtcgcacggcgcatggtgtggacatttatgccaaccatgaagtcctcccggaccccaacaaccgtcttgttctctccaaagaccataaggatgcgctcggtattccacatcctgaagtcacctacgatgttggggagtatgttcgaaagtcagctgtcatctcacgacagcgcctgatggatatcgccaaagccatgggcggtacggaaatcgagatgactccgtattttacgcccaacaaccacatcaccggtggcactatcatgggccatgatccacgggattcagtggtcgataaatggctccggacccatgatcattccaatctgttccttgcaacaggcgccacaatggcagcgtccggtacggtcaattcaacgttaacaatggccgcactgtcattacgcgcggcagatgccattctcaatgacctgaaacaggggtacgtaggcgcgccctggagccacccgcagttcgaaaaataataagcttgacctgtgaagtgaaaaatggcgcacattgtgcgacattttttttgtctgccgtttaccgctactgcgtcacggatctccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggat

SEQ ID NO: 18 - DNA, 6986 bp, artificial
  plasmid pBBR1p264-sacC-strep
ctcgggccgtctcttgggcttgatcggccttcttgcgcatctcacgcgctcctgcggcggcctgtagggcaggctcatacccctgccgaaccgcttttgtcagccggtcggccacggcttccggcgtctcaacgcgctttgagattcccagcttttcggccaatccctgcggtgcataggcgcgtggctcgaccgcttgcgggctgatggtgacgtggcccactggtggccgctccagggcctcgtagaacgcctgaatgcgcgtgtgacgtgccttgctgccctcgatgccccgttgcagccctagatcggccacagcggccgcaaacgtggtctggtcgcgggtcatctgcgctttgttgccgatgaactccttggccgacagcctgccgtcctgcgtcagcggcaccacgaacgcggtcatgtgcgggctggtttcgtcacggtggatgctggccgtcacgatgcgatccgccccgtacttgtccgccagccacttgtgcgccttctcgaagaacgccgcctgctgttcttggctggccgacttccaccattccgggctggccgtcatgacgtactcgaccgccaacacagcgtccttgcgccgcttctctggcagcaactcgcgcagtcggcccatcgcttcatcggtgctgctggccgcccagtgctcgttctctggcgtcctgctggcgtcagcgttgggcgtctcgcgctcgcggtaggcgtgcttgagactggccgccacgttgcccattttcgccagcttcttgcatcgcatgatcgcgtatgccgccatgcctgcccctcccttttggtgtccaaccggctcgacgggggcagcgcaaggcggtgcctccggcgggccactcaatgcttgagtatactcactagactttgcttcgcaaagtcgtgaccgcctacggcggctgcggcgccctacgggcttgctctccgggcttcgccctgcgcggtcgctgcgctcccttgccagcccgtggatatgtggacgatggccgcgagcggccaccggctggctcgcttcgctcggcccgtggacaaccctgctggacaagctgatggacaggctgcgcctgcccacgagcttgaccacagggattgcccaccggctacccagccttcgaccacatacccaccggctccaactgcgcggcctgcggccttgccccatcaatttttttaattttctctggggaaaagcctccggcctgcggcctgcgcgcttcgcttgccggttggacaccaagtggaaggcgggtcaaggctcgcgcagcgaccgcgcagcggcttggccttgacgcgcctggaacgacccaagcctatgcgagtgggggcagtcgaaggcgaagcccgcccgcctgccccccgagcctcacggcggcgagtgcgggggttccaagggggcagcgccaccttgggcaaggccgaaggccgcgcagtcgatcaacaagccccggaggggccactttttgccggagggggagccgcgccgaaggcgtgggggaaccccgcaggggtgcccttctttgggcaccaaagaactagatatagggcgaaatgcgaaagacttaaaaatcaacaacttaaaaaaggggggtacgcaacagctcattgcggcaccccccgcaatagctcattgcgtaggttaaagaaaatctgtaattgactgccacttttacgcaacgcataattgttgtcgcgctgccgaaaagttgcagctgattgcgcatggtgccgcaaccgtgcggcaccctaccgcatggagataagcatggccacgcagtccagagaaatcggcattcaagccaagaacaagcccggtcactgggtgcaaacggaacgcaaagcgcatgaggcgtgggccgggcttattgcgaggaaacccacggcggcaatgctgctgcatcacctcgtggcgcagatgggccaccagaacgccgtggtggtcagccagaagacactttccaagctcatcggacgttctttgcggacggtccaatacgcagtcaaggacttggtggccgagcgctggatctccgtcgtgaagctcaacggccccggcaccgtgtcggcctacgtggtcaatgaccgcgtggcgtggggccagccccgcgaccagttgcgcctgtcggtgttcagtgccgccgtggtggttgatcacgacgaccaggacgaatcgctgttggggcatggcgacctgcgccgcatcccgaccctgtatccgggcgagcagcaactaccgaccggccccggcgaggagccgcccagccagcccggcattccgggcatggaaccagacctgccagccttgaccgaaacggaggaatgggaacggcgcgggcagcagcgcctgccgatgcccgatgagccgtgttttctggacgatggcgagccgttggagccgccgacacgggtcacgctgccgcgccggtagcacttgggttgcgcagcaacccgtaagtgcgctgttccagactatcggctgtagccgcctcgccgccctataccttgtctgcctccccgcgttgcgtcgcggtgcatggagccgggccacctcgacctgaatggaagccggcggcacctcgctaacggattcaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtcggcctattggttaaaaaatgagctgatttaacaaaaatttaacgcgaattttaacaaaatattaacgcttacaatttccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattggagctcgttgcgctgaatgagaggaaagccgtggcgagaagtgccaggccaaggatgacataatagagagggccgccgatggcagcgacccatgcgccgccaacggtaaggatcgcgccgcaaagcgcgaataccgcggccgtaatcagggcccagagccctggccgggatgttgtgctcatgatgttcctggatctgttgttctgatgcatcacggtgacttccggacaaagcacatggatgggcgtcaagggtcaagatgtcagaaggttcatcttgctcacgtcactgtgaggactttcacgtcgtcgtgacttgaggaaaggaaggacttatgattgagcgttcaagaaaataatgcgcttgcaggccacttgggcttacgataaggacaccgcttccgcgcacgcgtccgggccacgagtggcatgcccggccgtgcagagtctctgcaggatgcaggaccaatcgtttacggcgagggagaccgaatttgggctgcaggaattcgatatcaagcttatcgataccgtcgacctcgaggtttaactttaagaaggagatatacatatgaaatacctgctgccgaccgctgctgctggtctgctgctcctcgctgcccagccggcgatggcctacgtatttaattttaatgccagtcgctggacgcgagcccaagcgatgaaagtgaataaatttgatttgacgacctctatgccggaaatcggcactgattttcctattatgcgtgatgacttgtggctgtgggatacttggccattacgggatatcaatggcaatcctgtcagctttaaaggctggaatgttattttctctttggtcgctgaccgcaatattccgtggaatgatcgccattctcatgcccgcatcggctatttctattctaaagatggtaaaagctgggtttatggtggccatctattgcaagaattggccaatagccgcacggcagaatggtccggcggcacgattatggcaccgggttcccgtaatcaggtcgaaaccttctttacctcgactttattcgacaagaatggcgtcagagaagccgttgctgctgttacgaaaggccgcatttatgcggatagtgaaggcgtttggttcaagggttttgaccagtcaacggatttgtttcaggctgatggtctgttttatcaaaattatgcagaaaataatctctggaatttccgtgatcctcatgttttcatcaatcctgaggatggtgagacctatgctttgttcgaagccaatgttgccactgtccgtggagaagacgatataggcgaggatgaaattggccctgttccggccaatacggtcgttccaaaagatgccaatttatgttcggcctctattggtattgcgcgttgtttgtcgccggatcgcaccgaatgggagctgttaccgcctttgttgacagcctttggcgtcaatgaccagatggaacggcctcatgtcattttccagaatggtttgacctatctctttacgatcagccatgattcgacttatgccgatggcttaaccggttccgatggtctttatggctttgtttccgaaaatggtatttttggcccctatgagccgctgaatggttctggccttgttctcggtggccctgcgtcacagccgaccgaagcctatgctcattacatcatgaataatggcttggttgaatcttttatcaatgaaatcattgatcccaaaagcggcaaagtcattgcgggcggtagcttggcaccgacggttcgcgttgaattacagggacatgagactttcgcgaccgaagtctttgattatggatatattcccgcatcttatgcttggccggtatggcctttcccggatcgtcgcaaaggcgcgcccggtggaggttcagcttggagccacccgcagttcgaaaaataataagcttgacctgtgaagtgaaaaatggcgcacattgtgcgacattttttttgtctgccgtttaccgctactgcgtcacggatctccacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcggattaatgaatcggccaacgcgcggggagaggcggtttgcgtattgggcgcatgcataaaaactgttgtaattcattaagcattctgccgacatggaagccatcacaaacggcatgatgaacctgaatcgccagcggcatcagcaccttgtcgccttgcgtataatatttgcccatgggggtgggcgaagaactccagcatgagatccccgcgctggaggatcatccagccggcgtcccggaaaacgattccgaagcccaacctttcatagaaggcggcggtggaatcgaaatctcgtgatggcaggttgggcgtcgcttggtcggtcatttcgaaccccagagtcccgctcagaagaactcgtcaagaaggcgatagaaggcgatgcgctgcgaatcgggagcggcgataccgtaaagcacgaggaagcggtcagcccattcgccgccaagctcttcagcaatatcacgggtagccaacgctatgtcctgatagcggtccgccacacccagccggccacagtcgatgaatccagaaaagcggccattttccaccatgatattcggcaagcaggcatcgccatgggtcacgacgagatcctcgccgtcgggcatgcgcgccttgagcctggcgaacagttcggctggcgcgagcccctgatgctcttcgtccagatcatcctgatcgacaagaccggcttccatccgagtacgtgctcgctcgatgcgatgtttcgcttggtggtcgaatgggcaggtagccggatcaagcgtatgcagccgccgcattgcatcagccatgatggatactttctcggcaggagcaaggtgagatgacaggagatcctgccccggcacttcgcccaatagcagccagtcccttcccgcttcagtgacaacgtcgagcacagctgcgcaaggaacgcccgtcgtggccagccacgatagccgcgctgcctcgtcctgcagttcattcagggcaccggacaggtcggtcttgacaaaaagaaccgggcgcccctgcgctgacagccggaacacggcggcatcagagcagccgattgtctgttgtgcccagtcatagccgaatagcctctccacccaagcggccggagaacctgcgtgcaatccatcttgttcaatcatgcgaaacgatcctcatcctgtctcttgatcagatcttgatcccctgcgccatcagatccttggcggcaagaaagccatccagtttactttgcagggcttcccaaccttaccagagggcgccccagctggcaattccggttcgcttgctgtccataaaaccgcccagtctagctatcgccatgtaagcccactgcaagctacctgctttctctttgcgcttgcgttttcccttgtccagatagcccagtagctgacattcatcccaggtggcacttttcggggaaatgtgcgcgcccgcgttcctgctggcgctgggcctgtttctggcgctggacttcccgctgttccgtcagcagcttttcgcccacggccttgatgatcgcggcggccttggcctgcatatcccgattcaacggccccagggcgtccagaacgggcttcaggcgctcccgaaggt